(19) United States
(12) Patent Application Publication
(10) Pub. No.: US 2024/0206787 A1
(43) Pub. Date: Jun. 27, 2024

(54) Dermal Patch with Biometric Sensor

(71) Applicant: Satio, Inc., Boston, MA (US)

(72) Inventors: Namal NAWANA, Weston, MA (US); Ziad Tarik AL-SHAMSIE, San Diego, CA (US); Mike MONIZ, Boston, MA (US)

(73) Assignee: Satio, Inc., Boston, MA (US)

(21) Appl. No.: 18/596,098

(22) Filed: Mar. 5, 2024

Related U.S. Application Data
(63) Continuation-in-part of application No. 18/090,063, filed on Dec. 28, 2022.
Application No. 18/090,063 is a continuation-in-part of application No. 17/903,802, filed on Sep. 6, 2022.
Application No. 17/903,802 is a continuation-in-part of application No. 17/500,873, filed on Oct. 13, 2021, now Pat. No. 11,964,121.
Application No. 17/500,873 is a continuation-in-part of application No. 17/994,454, filed on Nov. 28, 2022, now Pat. No. 11,877,848.
Application No. 17/994,454 is a continuation-in-part of application No. 17/971,142, filed on Oct. 21, 2022.
Application No. 17/971,142 is a continuation-in-part of application No. 17/991,284, filed on Nov. 21, 2022.
Application No. 17/991,284 is a continuation-in-part of application No. 17/971,142, filed on Oct. 21, 2022.
Application No. 17/971,142 is a continuation-in-part of application No. 17/521,466, filed on Nov. 8, 2021, now Pat. No. 11,510,602.
Application No. 17/903,802 is a continuation-in-part of application No. 17/412,213, filed on Aug. 25, 2021, now Pat. No. 11,452,474.
Application No. 18/090,063 is a continuation-in-part of application No. 63/174,956, filed on Apr. 14, 2021.
Application No. 63/174,956 is a continuation-in-part of application No. 18/090,026, filed on Dec. 28, 2022.
Application No. 18/090,026 is a continuation-in-part of application No. 17/903,802, filed on Sep. 6, 2022.
Application No. 17/903,802 is a continuation-in-part of application No. 17/500,873, filed on Oct. 13, 2021, now Pat. No. 11,964,121.
Application No. 17/500,873 is a continuation-in-part of application No. 17/994,454, filed on Nov. 28, 2022, now Pat. No. 11,877,848.
Application No. 17/994,454 is a continuation-in-part of application No. 17/971,142, filed on Oct. 21, 2022.
Application No. 17/971,142 is a continuation-in-part of application No. 17/991,284, filed on Nov. 21, 2022.
Application No. 17/994,454 is a continuation-in-part of application No. 17/719,881, filed on Apr. 13, 2022.
Application No. 17/719,881 is a continuation-in-part of application No. 17/412,205, filed on Aug. 25, 2021.
(60) Provisional application No. 63/190,700, filed on May 19, 2021.
(60) Provisional application No. 63/174,956, filed on Apr. 14, 2021.
(60) Provisional application No. 63/174,956, filed on Apr. 14, 2021.
(60) Provisional application No. 63/190,700, filed on May 19, 2021.
(60) Provisional application No. 63/174,956, filed on Apr. 14, 2021.

(51) Int. Cl. A61B 5/157; A61B 5/00; A61B 5/1172; A61B 5/15; A61B 5/151

(52) U.S. Cl. CPC A61B 5/157; A61B 5/1172; A61B 5/150267; A61B 5/15194; A61B 5/6833; A61B 2560/0214; A61B 2562/227

(57) ABSTRACT

A dermal patch system for collecting a physiological sample from a subject includes a cartridge configured to attach to the skin of the subject, and a lancet configured to engage with the cartridge and draw a physiological sample upon engagement. The cartridge includes a biometric sensor configured to obtain biometric data from a subject.

### RELATED APPLICATIONS

The present application claims priority to U.S. application Ser. No. 17/719,881 filed on Apr. 13, 2022, which claims priority to U.S. Provisional Patent Application No. 63/174,956 filed on Apr. 14, 2021, U.S. application Ser. No. 17/412,205 filed on Aug. 25, 2021, which claims priority to U.S. Provisional Patent Application No. 63/190,700 filed on May 19, 2021 and U.S. Provisional Patent Application No. 63/174,956 filed on Apr. 14, 2021, U.S. application Ser. No. 17/747,544 filed on May 18, 2022, which claims priority to U.S. Provisional Patent Application No. 63/190,700 filed on May 19, 2021, U.S. application Ser. No. 17/903,802 filed on Sep. 6, 2022, U.S. application Ser. No. 18/090,026 filed on Dec. 28, 2022, U.S. application Ser. No. 17/971,142 filed on Oct. 21, 2022, U.S. application Ser. No. 17/991,284 filed on Nov. 21, 2022 and U.S. application Ser. No. 18/090,063 filed on Dec. 28, 2022, which are hereby incorporated by reference herein in their entireties.

### TECHNICAL FIELD

The present teachings are generally directed to a lancet for use with a cartridge of a dermal patch system (also referred to as “a dermal patch” herein) and a dermal patch system that includes a biometric sensor for identification of a subject from whom a physiological sample is drawn for analysis.

### BACKGROUND

Several diagnostic tests may be performed by analyzing a physiological sample from a subject. In order to obtain the physiological sample, a subject may have to travel to a diagnostic laboratory, where a medical professional obtains the sample with a standard syringe. This process may be time consuming and burdensome to the subject.

### SUMMARY

Aspects of the present disclosure address the above-referenced problems and/or others.

In one aspect, a dermal patch system for collecting a physiological sample from a subject includes a cartridge configured to attach to the skin of the subject and a lancet configured to engage with the cartridge and draw a physiological sample upon engagement. The cartridge includes a biometric sensor, such as a fingerprint scanner, configured to obtain biometric data, e.g., fingerprint data, from a subject. In some embodiments, the cartridge further includes a computer system configured to receive the biometric data, e.g., the fingerprint data, from the biometric scanner and store the biometric data within a memory of the computer system. In some embodiments, the cartridge further includes a power supply configured to power the biometric sensor and the computer system. In some embodiments, the power supply is configured to be activated to supply electrical power to the biometric sensor and the computer system when the lancet engages with the cartridge. By way of example, in some embodiments, in absence of the engagement of the lancet with the cartridge, the electrical connection between the power supply and the biometric sensor is in the form of an open circuit such that the engagement of the lancet with the cartridge closes the electrical circuit to allow the power supply to provide electrical power to the biosensor and the computer system In some such embodiments, the cartridge includes first electrical contacts and the lancet includes second electrical contacts, wherein the engagement of the lancet with the cartridge generates an electrical path between the first and second electrical contacts to close the circuit, e.g., the engagement of the lancet with the cartridge can result in physical contact between. By way of example, in some embodiments, the first and second electrical contacts are pogo style connectors. In some embodiments, the first electrical contacts are electrically conductive pads and the second electrical contacts are pogo style connectors. In some such embodiments, the pogo style connectors of the lancet can engage with the conductive pads of the cartridge when the lancet is received by the cartridge.

In some embodiments, the lancet includes a needle and the cartridge includes a lancet receiving element that is configured to automatically cause the lancet to deploy the needle to draw a physiological sample upon engagement of the cartridge with the lancet. In some embodiments, the lancet is configured to automatically retract the needles. In some embodiments, the lancet receiving element includes the first electrical contacts and a portion of the lancet that engages with the lancet receiving element includes the second electrical contacts. In some embodiments, the power supply is a battery or a charged capacitor. In some embodiments, the power supply is a charged capacitor that automatically powers the biometric sensor and the computer system when the lancet engages with the cartridge. In some embodiments, the cartridge is configured to receive and store the physiological sample.

In another aspect, a method for obtaining a physiological sample from a subject includes affixing a cartridge of a dermal patch system to the subject's skin, engaging a lancet with the cartridge to draw a physiological sample from the subject, and using a biometric sensor, e.g., a fingerprint scanner, of the cartridge to obtain biometric data of the subject from whom the physiological sample is drawn. In some embodiments, the method further includes storing the drawn physiological sample within the cartridge. In some embodiments, the method also includes storing the biometric data in a memory of a computer system that is disposed within the cartridge. In some embodiments, the cartridge includes a power supply that is configured to power the biometric sensor and the computer system upon engagement of the lancet with the cartridge. In some embodiments, the power supply is a charged capacitor and engaging the lancet with the cartridge closes a circuit which causes the charged capacitor to automatically power the biometric sensor and the computer system. In some embodiments, the method also includes determining if chain of custody can be established by comparing the fingerprint data stored in the memory of the computer system to other fingerprint data. In certain applications, such as drug testing, it is desirable to validate the chain of custody of a sample under analysis

### BRIEF DESCRIPTION OF THE DRAWINGS

Aspects of the present disclosure may take form in various components and arrangements of components, and in various steps and arrangements of steps. The drawings are only for illustration purpose of preferred embodiments of the present disclosure and are not to be considered as limiting.

Features of embodiments of the present disclosure will be more readily understood from the following detailed description take in conjunction with the accompanying drawings in which:

FIG. 1 depicts a dermal patch system in accordance with an exemplary embodiment of the present disclosure;

FIG. 2 depicts a lancet with three needles in accordance with an exemplary embodiment of the present disclosure;

FIG. 3 is a cross sectional view of the lancet, wherein the lancet is in an undeployed position in accordance with an exemplary embodiment of the present disclosure;

FIG. 4 is a cross sectional view of a housing of the lancet in accordance with an exemplary embodiment of the present disclosure;

FIG. 5 is a cross sectional view of a housing of the lancet in accordance with an exemplary embodiment of the present disclosure;

FIG. 6 is a cross sectional view of a housing of the lancet in accordance with an exemplary embodiment of the present disclosure;

FIG. 7 is a bottom view of a housing of the lancet in accordance with an exemplary embodiment of the present disclosure;

FIG. 8 is a cross sectional view of a cap of the lancet in accordance with an exemplary embodiment of the present disclosure;

FIG. 9 depicts a side of an inner sleeve of the lancet in accordance with an exemplary embodiment of the present disclosure;

FIG. 10 depicts another side of the inner sleeve of the lancet in accordance with an exemplary embodiment of the present disclosure;

FIG. 11 is a cross sectional view of the inner sleeve of the lancet in accordance with an exemplary embodiment of the present disclosure;

FIG. 12 depicts a side of a needle platform of the lancet in accordance with an exemplary embodiment of the present disclosure;

FIG. 13 depicts another side of the needle platform in accordance with an exemplary embodiment of the present disclosure;

FIG. 14 is a cross sectional view of a spring sleeve of the lancet in accordance with an exemplary embodiment of the present disclosure;

FIG. 15 is a top view of a cover of a cartridge of the dermal patch system in accordance with an exemplary embodiment of the present disclosure;

FIG. 16 is a cross sectional view of the cover in accordance with an exemplary embodiment of the present disclosure;

FIG. 17 is a top view of a base of the cartridge of the dermal patch system in accordance with an exemplary embodiment of the present disclosure;

FIG. 18 is a cross sectional view of a lancet receiving element of the base in accordance with an exemplary embodiment of the present disclosure;

FIG. 19 is a top view of a base of the cartridge of the dermal patch system in accordance with an exemplary embodiment of the present disclosure;

FIG. 20 is a cross sectional view of a lancet receiving element of the base in accordance with an exemplary embodiment of the present disclosure;

FIG. 21 is a cross sectional view of a lancet receiving element of the base in accordance with an exemplary embodiment of the present disclosure;

FIG. 22 is a cross sectional view of a lancet receiving element of the base in accordance with an exemplary embodiment of the present disclosure;

FIG. 23 is another cross sectional view of a lancet receiving element of the base in accordance with an exemplary embodiment of the present disclosure;

FIG. 24 is a cross sectional view of the base in accordance with an exemplary embodiment of the present disclosure;

FIG. 25 schematically depicts a physiological sample well, a physiological sample channel, and a sample collection reservoir of the base in accordance with an exemplary embodiment of the present disclosure;

FIG. 26 is a cross sectional view of the lancet engaged with the base, wherein the needle platform is in a released position in accordance with an exemplary embodiment of the present disclosure;

FIG. 27 is another cross sectional view of the lancet engaged with the base, wherein the needle platform is in a released position in accordance with an exemplary embodiment of the present disclosure;

FIG. 28 is a cross sectional view of the lancet, wherein the needle platform is in a released position in accordance with an exemplary embodiment of the present disclosure;

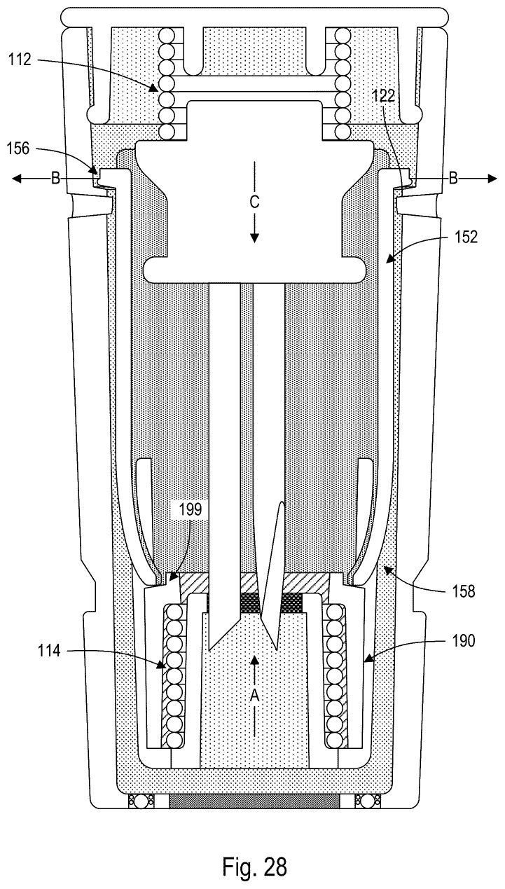

FIG. 29 is a cross sectional view of the lancet transitioning to a deployed position in accordance with an exemplary embodiment of the present disclosure;

FIG. 30 is a cross sectional view of the lancet engaged with the base, wherein the lancet is in a deployed position in accordance with an exemplary embodiment of the present disclosure;

FIG. 31 is a cross sectional view of the lancet, wherein the lancet is in a deployed position in accordance with an exemplary embodiment of the present disclosure;

FIG. 32 is a cross sectional view of the lancet engaged with the base, wherein the lancet is in a retracted position in accordance with an exemplary embodiment of the present disclosure;

FIG. 33 is a cross sectional view of the lancet, wherein the lancet is in a retracted position in accordance with an exemplary embodiment of the present disclosure;

FIG. 34 is a flowchart of a method for obtaining a physiological sample from a subject;

FIG. 35 schematically depicts a computer system in accordance with an exemplary embodiment of the present disclosure; and

FIG. 36 schematically depicts a cloud computing environment in accordance with an exemplary embodiment.

### DETAILED DESCRIPTION

The present disclosure generally relates to a dermal patch system (which may also be referred to as a “dermal patch”) that may be utilized to collect and optionally store a physiological sample.

In various aspects, the present teachings address an unmet need for devices that allow efficient, easy and reliable extraction of biological samples from a subject.

In various embodiments, a dermal patch system is disclosed that allows collecting a physiological sample and storing the collected physiological sample, e.g., on a sample collection pad and/or a reservoir provided on the dermal patch system. Dermal patch systems disclosed herein may allow for the collection and analysis of a physiological sample in a variety of environments (e.g., at home, in the field, in a medical facility, etc.).

The term “about,” as used herein, denotes a deviation of at most 10% relative to a numerical value. For example, about 100 μm means in the range of 90 μm-110 μm.

The term “substantially,” as used herein, refers to a deviation, if any, of at most 10% from a complete state and/or condition.

The term “majority,” as used herein, refers to more than 50% of an object or substance.

The term “subject” as used herein refers to a human subject or an animal subject (e.g., chicken, pig, cattle, dog, cat, etc.).

The term “transparent,” as used herein, indicates that light can substantially pass through an object (e.g., a window) to allow visualization of a material disposed behind the object. For example, in some embodiments, a transparent object allows the passage of at least 70%, or at least 80%, or at least 90% of visible light therethrough.

The term “needle” as used herein, refers to a component with a pointed tip that is configured to pierce an outer surface of an element (e.g., skin of a subject) to provide a passageway through which a physiological fluid can be extracted.

The present disclosure generally relates to a device, which is herein also referred to as a dermal patch or a dermal patch system, for collecting a physiological sample (e.g., bodily fluids such as blood, interstitial fluids, etc.) from a subject. In some embodiments discussed below, such a dermal patch system can include a cartridge that can be affixed to a subject's skin (e.g., via an adhesive layer) and a separate lancet that can be engaged with the cartridge to puncture the skin, thereby providing a passageway for extracting the physiological sample. As discussed in more detail below, the lancet can include a housing in which at least one needle that is configured for puncturing the skin is disposed. The lancet can be transitioned between at least three states. In a first state (which may be referred to as an undeployed or a locked state) the lancet retains the needle(s) within the lancet housing when the lancet is not engaged with the cartridge. In a second state (which may be referred to as a deployed, an unlocked state, or a released state) the lancet allows the needle(s) to be deployed to allow the needle(s) to exit the housing, so as to be available for puncturing the skin in response to engagement of the lancet with the cartridge. In a third state (which may be referred to as a retracted state), subsequent to the deployment of the needle(s), the lancet automatically moves the needle(s) from the deployed position back into the lancet housing. In other words, the engagement of the lancet with the cartridge automatically transitions the needle(s) from the locked state to the unlocked state, and to the retracted state.

In some embodiments, as discussed in more detail below, the lancet includes an inner sleeve with a plurality of deformable tabs and a spring sleeve. In a locked state, the deformable tabs can apply a pressure to the spring sleeve to maintain the spring sleeve in a first position in which the spring sleeve compresses the lower spring into a compressed state and prevents the lower spring from expanding.

When the lancet is engaged with the cartridge, the engagement of the lancet with the cartridge causes the inner sleeve to move from a first position to a second position. In the first position, the inner sleeve retains a needle platform that supports the needles in an undeployed position. The lancet also includes an upper spring that is coupled to the needle platform. In the undeployed position, the needle platform compresses the upper spring and prevents the upper spring form expanding. In the second position, the inner sleeve moves vertically upward, which releases the needle platform thereby allowing the needle platform to move to a deployed position. When released, the upper spring is allowed to expand thereby applying a force to the needle platform, which causes the movement of the needle platform such that the needles extend beyond the housing of the lancet and become available for puncturing the skin.

Furthermore, this force causes the needles (which are coupled to the needle platform) to travel with sufficient velocity to puncture the skin of a subject. As the needle platform travels to the deployed position, the needle platform contacts the deformable tabs and causes the tabs to break or flex, e.g., to be deflected away from the lancet's longitudinal axis. When broken (or deflected), the tabs no longer contact the spring sleeve, or apply a lower force onto the spring sleeve, thereby allowing the lower spring to expand and move the spring sleeve vertically upward from a first position to a second position, which in turn moves the needle spring vertically upward such that the needles are retracted into the lancet's housing.

In this manner, the lancet remains safe before it is engaged with the cartridge as the needles in the lancet cannot be deployed when the lancet is not engaged with the cartridge. Furthermore, in this manner, the lancet remains safe after drawing a physiological sample as the needles automatically retract back into the lancet's housing after being deployed.

The cartridge can include a biometric sensor (e.g., a fingerprint scanner, iris scanner, etc.) and a computer system (e.g., a system on a chip “SOC”) that is in communication with the biometric sensor. The biometric sensor is configured to obtain biometric data (e.g., fingerprint data, iris data) from a user of the dermal patch (e.g., a subject from whom a physiological sample is extracted, another user attaching the dermal patch to the subject, a medical professional, etc.). The biometric sensor is further configured to transmit the biometric data to the computer system. The computer system is configured to store the received biometric data in a memory. In this embodiment, the cartridge includes a power supply (e.g., a battery, a charged capacitor, etc.) that is in electrical communication with the biometric sensor and the computer system via an open circuit. The lancet and the cartridge each include electrical contacts that can engage with one another, e.g., automatically upon engagement of the lancet with the cartridge, to complete the circuit thereby allowing the power supply to power the biometric sensor and the computer system. That is, the biometric sensor can obtain biometric data of a subject only when the lancet is inserted into the cartridge attached to the subject's skin.

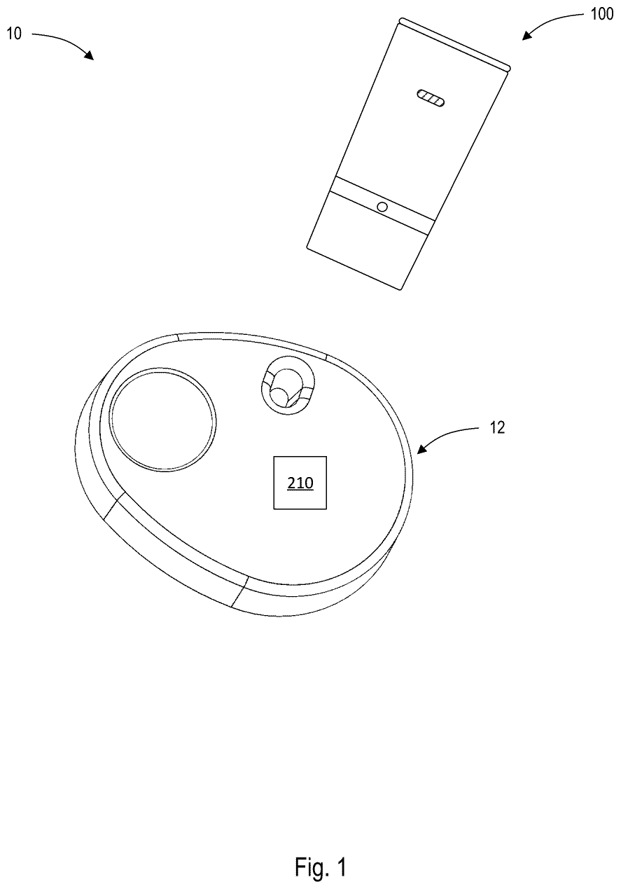

Referring now to FIG. 1, a dermal patch system 10 is shown in accordance with an exemplary embodiment. In this embodiment, the dermal patch system 10 includes a cartridge 12 that can be affixed to a subject's skin and a lancet 100 that can be engaged with the cartridge 12.

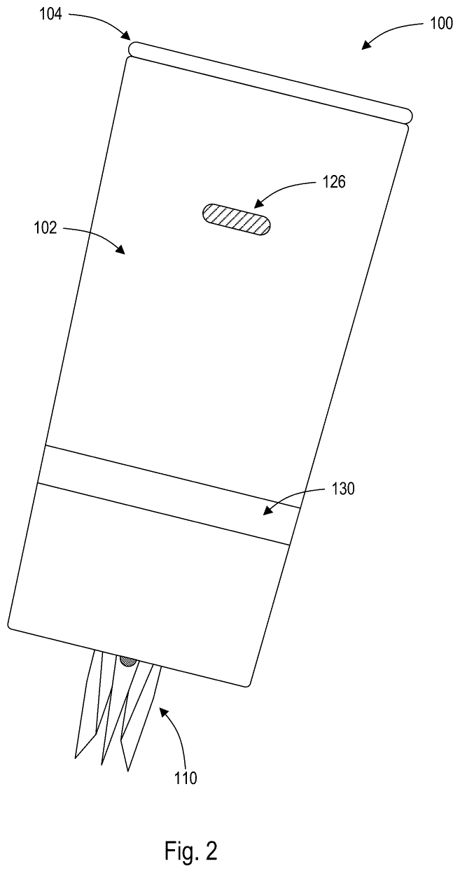

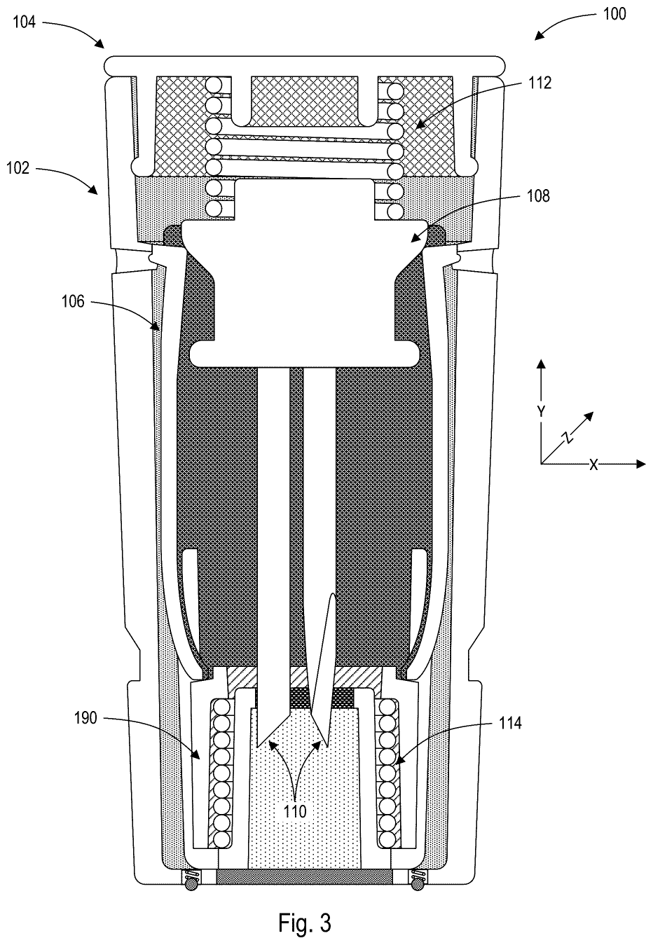

Referring to FIGS. 2 and 3, a cross sectional view of the lancet 100 is shown in accordance with an exemplary embodiment. The lancet 100 includes a housing 102 and a cap 104 that is coupled to the housing 102. The lancet extends longitudinally along an X-axis (FIG. 3) between a proximal end defined by the cap 104 and a distal end defined by an end of the housing 102 that is opposite the cap 104.

The housing 102 and the cap 104 define an inner volume of the lancet 100 in which various components of the lancet 100 are disposed. The lancet 100 further includes an inner sleeve 106 and a needle platform 108 that are disposed within the inner volume of the lancet 100. The needle platform 108 supports a plurality of needles 110, specifically three needles 110 in this embodiment.

While needle platform 108 is depicted as supporting three needles 110 (FIG. 2), in other embodiments, the needle platform 108 can support more or less needles 110 (e.g., 1, 2, 5, needles etc.). However, it has been discovered that the use of three needles is particularly advantageous in drawing blood from a subject. Further, in this embodiment, the needles have slanted ends with the bottom ends of the needles facing away from one another.

The lancet 100 further includes an injection spring (also referred to as an upper spring) 112 and a retraction spring (also referred to as a lower spring) 114 that can move the needle platform 108 in a manner discussed in more detail below.

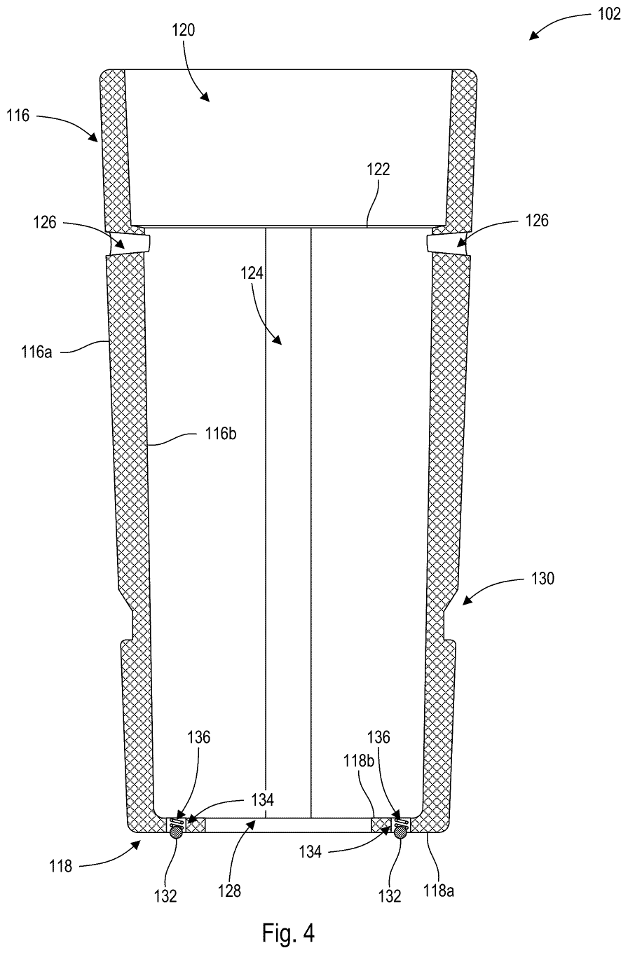

With particular reference to FIGS. 4-8, the housing 102 includes a side wall 116 and a bottom wall 118. The side wall 116 includes an outer surface 116a and an opposed inner surface 116b. The bottom wall 118 includes an outer surface 118a and an opposed inner surface 118b. The side wall 116 extends substantially vertically from the bottom wall 118. In this embodiment, the side wall 116 is substantially cylindrical in shape and the bottom wall 118 has a substantially circular cross-section. The side wall 116 and the bottom wall 118 are generally symmetric about a longitudinal axis (Y-axis) of the lancet 100.

The inner surface 116b of the side wall 116 and the inner surface 118b of the bottom wall 118 define an inner volume 120. The side wall 116 is tapered such that its inner diameter increases in a direction from its distal end to its proximal end such that the lancet housing exhibits a wider upper portion relative to its lower portion. Further, the inner surface 118b of the upper portion defines a ledge 122.

The inner surface 116b also defines a first and second longitudinal groove 124 that are opposed from one another (only one of which is shown in the cross section in FIG. 4). The grooves 124 extend between the ledge 122 and the inner surface 118b of the bottom wall 118.

The side wall 116 defines openings 126 that extend through the side wall 116. That is, the openings 126 extend between the outer surface 116a and the inner surface 116b of the side wall 116. The bottom wall 118 defines an aperture 128 that extends through the bottom wall 118. That is, the aperture 128 extends between the outer surface 118a and the inner surface 118b of the bottom wall 118. As will be discussed in further detail herein, when the lancet 100 is activated via engagement with the cartridge 12, the needles of the lancet 100 extend through the aperture 128 for puncturing a subject's skin to draw a physiological sample. The outer surface 116b of the side wall 116 further defines an outer groove 130. As will be discussed in further detail herein, a portion of the cartridge 12 engages with the outer groove 130 to couple the lancet 100 to the cartridge 12.

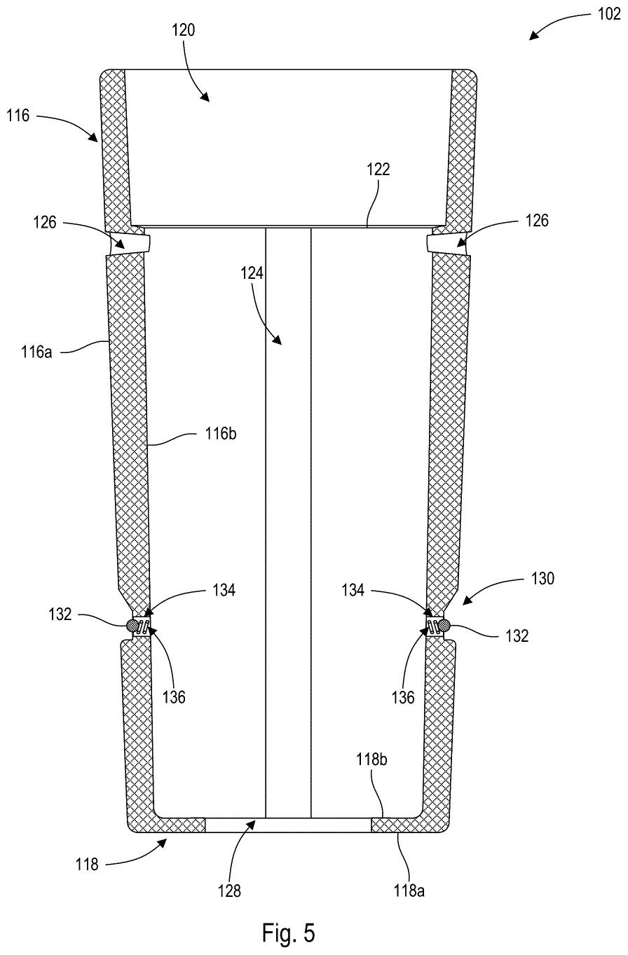

The housing 102 further includes a plurality of electrical contacts 132 are in electrical communication with one another and are formed of a material that is suitable for conducting electricity (e.g., copper, iron, gold, aluminum, etc.). In this embodiment, as depicted in FIGS. 4 and 5, the electrical contacts 132 are in the form of pogo style connectors and have a spherical shape. In one embodiment (FIG. 4), the bottom wall 118 includes contact receptacles 134 in each of which one of the electrical contacts 132 and a respective spring 136 can be housed.

When the lancet 100 is not engaged with the cartridge 12 the springs 136 are in a relaxed state and push the contacts 132 partially beyond the bottom wall 118. While FIG. 4 depicts the bottom wall 118 as including the electrical contacts 132, the contact receptacles 134, and the springs 136, these elements may be disposed elsewhere on the housing 102. For example, in another embodiment (FIG. 5), the groove 124 of the side wall 116 includes the contact receptacles 134 for housing the electrical contacts 132 and the respective springs 136. In this embodiment, when the lancet 100 is not engaged with the cartridge 12 the springs 136 are in a relaxed state and push the contacts 132 partially beyond the side wall 116. While the electrical contacts 132 are depicted as pogo style connectors, in other embodiments, the electrical contacts 132 may take other forms e.g., electrical contact pads).

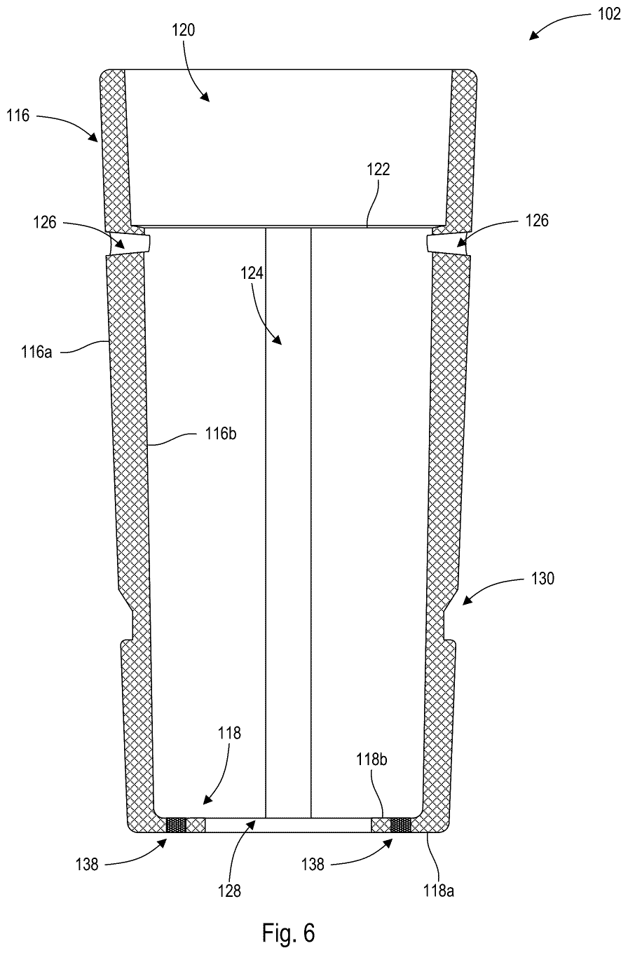

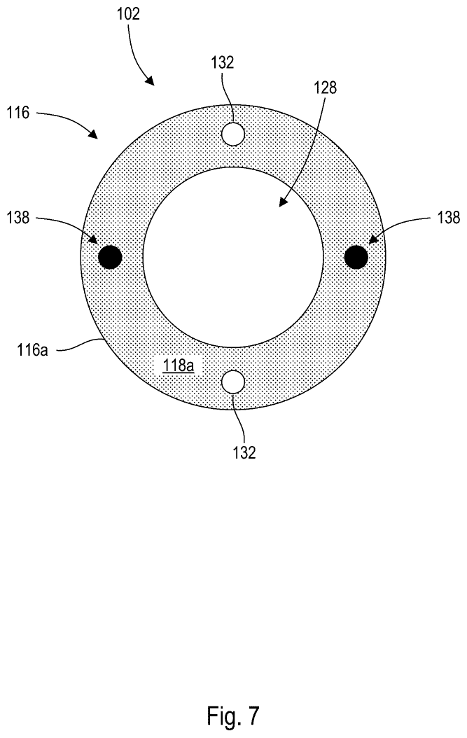

The bottom wall 118 further includes a plurality of openings 138 (FIGS. 6 and 7) that extends through bottom wall 118. That is, the openings 138 extend between the outer surface 118a and the inner surface 118b of the bottom wall 118. As will be discussed in further detail herein, the openings 138 aid in properly aligning the lancet 100 with the cartridge 12.

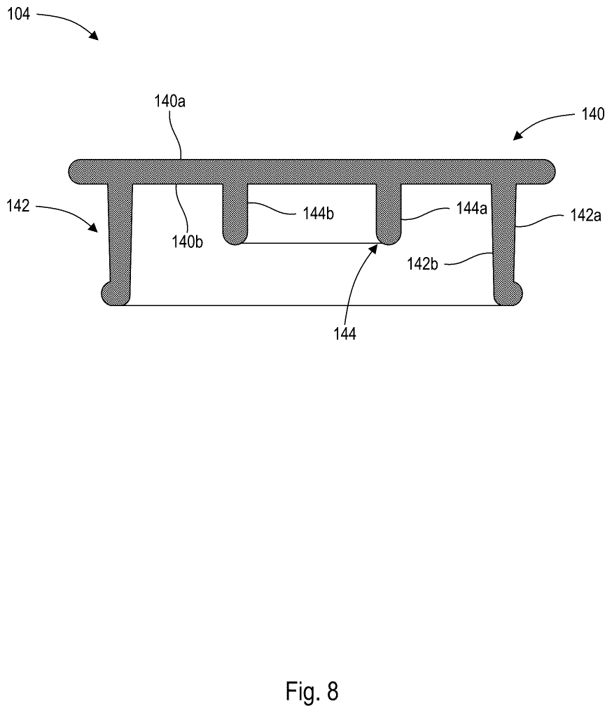

Referring now to FIG. 8, the inner cap 104 is shown in accordance with an exemplary embodiment. The cap 104 includes a top wall 140 with an outer surface 140a and an opposed inner surface 140b. The cap 104 also includes a side wall 142 with an outer surface 142a and an opposed inner surface 142b. The top wall 140 extends substantially horizontally from and perpendicular to the side wall 142. The side wall 142 extends substantially vertically from and perpendicular to the top wall 140. The top wall 140 and the side wall 142 have generally circular cross-sectional profiles and are concentric with one another. The cap 104 also includes an inner cylinder 144 with an outer surface 144a and an opposed inner surface 144b. The inner cylinder 144 extends substantially vertically from and perpendicular to the top wall 140. The inner cylinder 144 is generally concentric with the top wall 140 and the side wall 142. The inner cylinder 144 extends substantially vertically from and perpendicular to the top wall 140.

When the cap 104 is coupled to the housing 102 the side wall 142 extends into the inner volume 120 of the housing 102 and at least a portion of the side wall 142 contacts the inner surface 116b of the side wall 116 such that the cap 104 couples to the housing 102 via an interference fit.

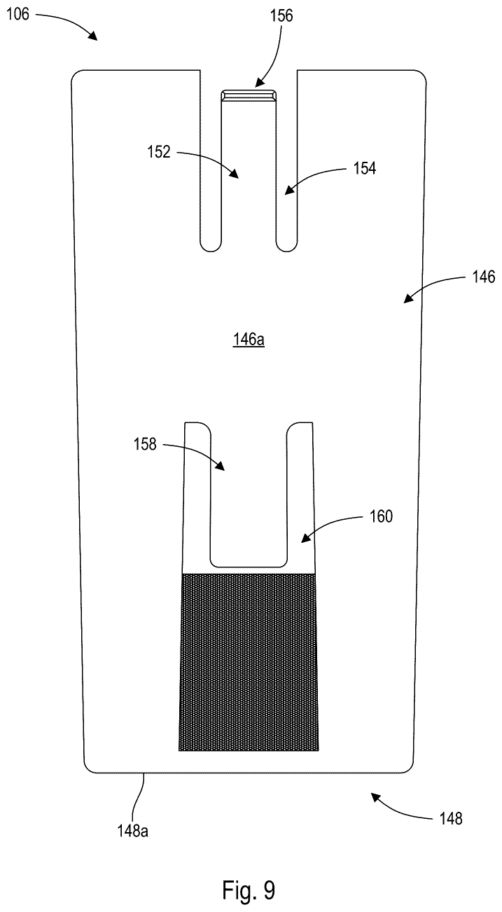

Referring now to FIGS. 9-11, the inner sleeve 106 is shown in accordance with an exemplary embodiment. The inner sleeve 106 includes a side wall 146 and a bottom wall 148. The side wall 146 includes an outer surface 146a and an opposed inner surface 146b. The bottom wall 148 includes an outer surface 148a and an opposed inner surface 148b. The side wall 146 extends substantially vertically from the bottom wall 148. The side wall 146 and the bottom wall 148 have generally circular cross-sectional profiles and are positioned concentrically relative to one another. The inner surface 146b of the side wall 146 and the inner surface 148b of the bottom wall 148 define an inner volume 150.

The side wall 146 defines a first and second deformable extension 152 that extend within a gap 154 of the side wall 146. The extensions 152 each include a hook 156. As will be discussed in further detail herein, the hooks 156 of the extensions 152 can be received by the openings 126 of the housing 102 to place the lancet 100 in the undeployed position.

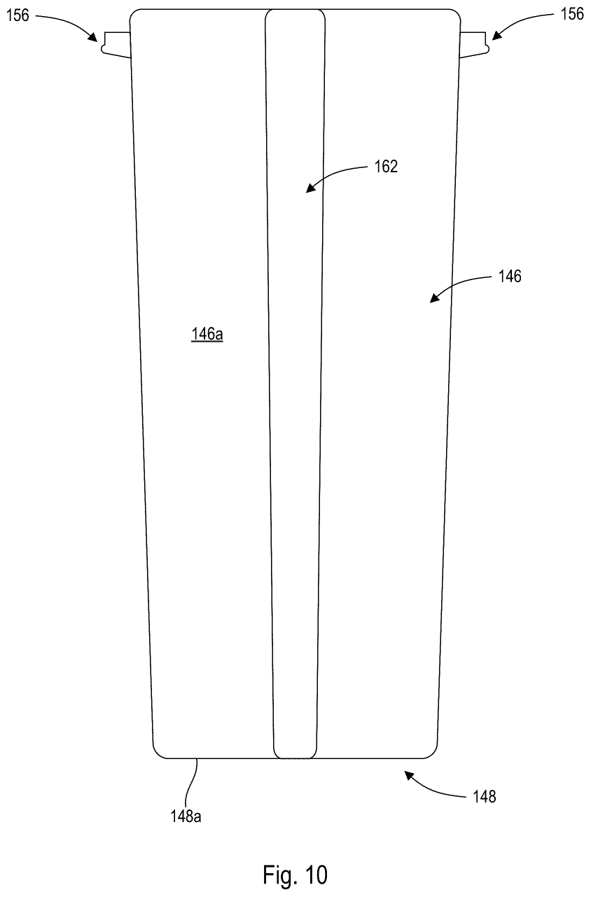

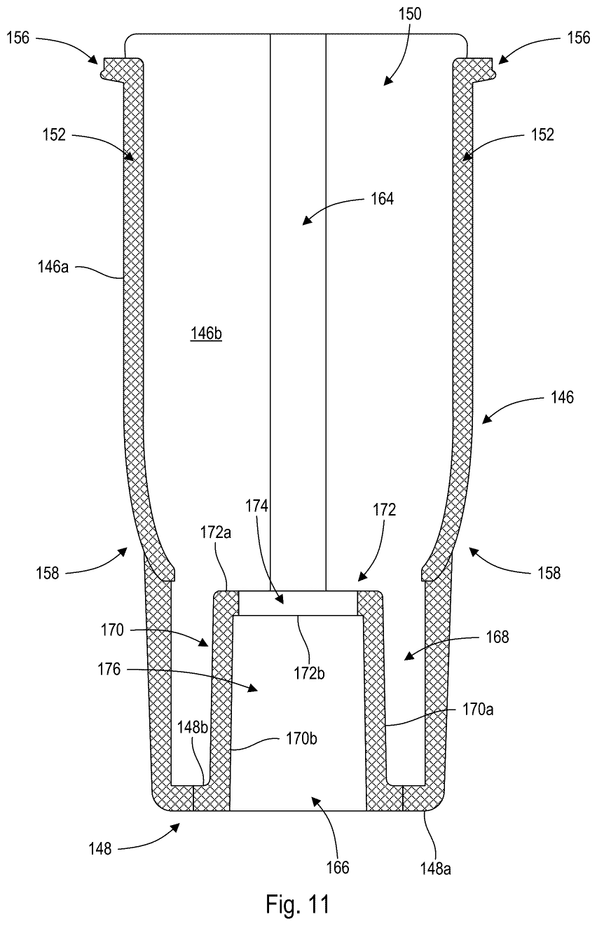

The side wall 146 further defines a first and second deformable tabs 158 that extend within an opening 160 of the side wall 146. The side wall 146 also includes a first and second protrusions 162 (only one of which is shown in FIG. 10) that extend substantially horizontally from and perpendicular to the outer surface 146a of the side wall 146. When the inner sleeve 106 is disposed within the housing 102, the protrusions 162 extend within the grooves 124 of the housing 102. When the inner sleeve 106 is moving between different positions (e.g., between an undeployed position to a deployed position), the grooves 124 and the protrusions 162 guide the vertical movement of the inner sleeve 106 while preventing a rotational or horizontal movement of the inner sleeve 106. The inner surface 146b of the side wall 146 further defines a first and second grooves 164 that are opposed from one another (only one of which is shown in FIG. 11). The grooves 164 extend substantially vertically from and perpendicular to the inner surface 148b of the bottom wall 148.

The inner sleeve 106 includes an opening 166 that extends through the bottom wall 148. That is, the opening 166 extends between the outer surface 148a and the inner surface 148b of the bottom wall 148. The inner sleeve 106 also includes a post 168 that is cylindrical in shape and is generally concentric with the opening 166. The post 168 includes a side wall 170 and a top wall 172. The side wall 170 extends substantially vertically from and perpendicular to the inner surface 148b of the bottom wall 148. The side wall 170 includes an outer surface 170a and an opposed inner surface 170b. The top wall 172 extends substantially horizontally between the side wall 170 and includes a top surface 172a and an opposed bottom surface 172b. The top wall 172 includes an opening 174 that extends through the top wall 172. That is, the opening 174 extends between the top surface 172a and the bottom surface 172b of the top wall 172. The inner surface 170b of the side wall 170 and the bottom surface 172b of the top wall 172 define an inner volume 176 of the post 168. As will be discussed in further detail herein, the needles 110 extend through the opening 174 of the top wall 172, through the inner volume 176, and through the opening 166 of the bottom wall 148 when in the deployed position.

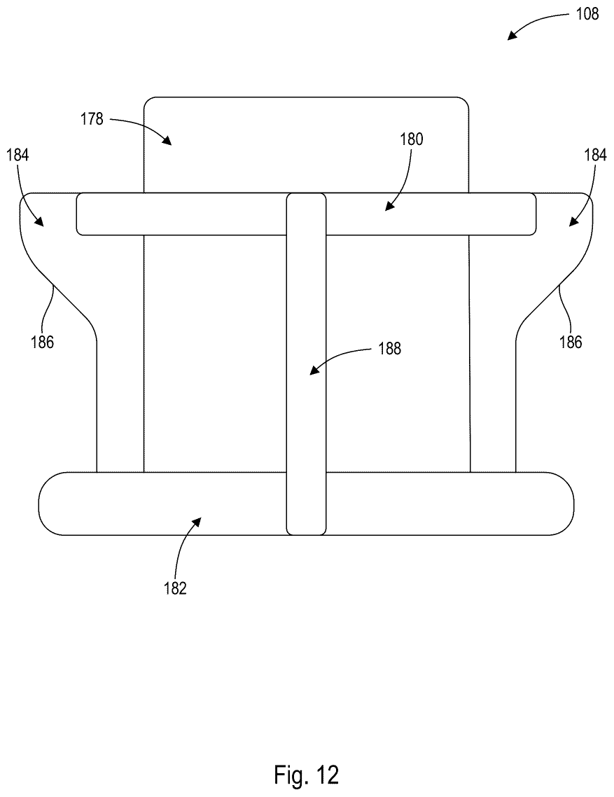

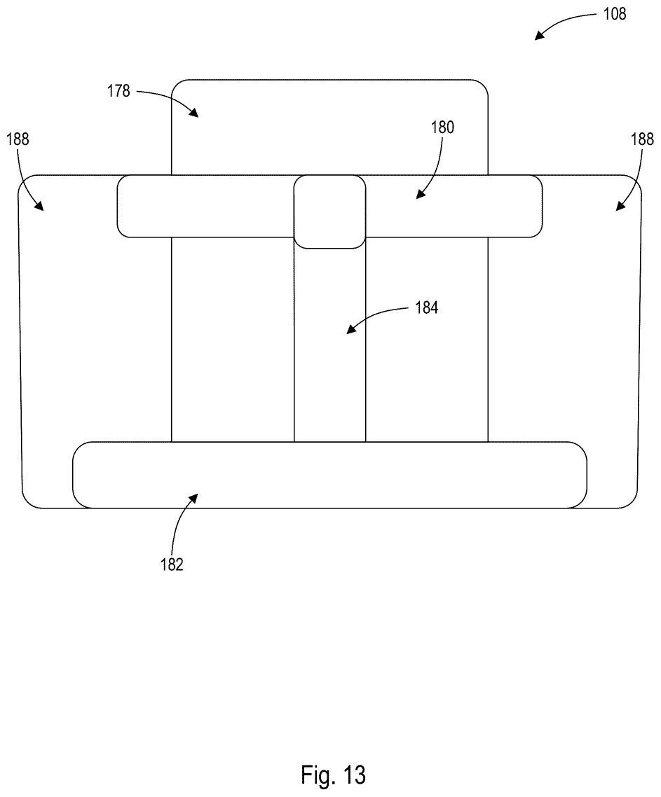

Referring now to FIGS. 12 and 13, the needle platform 108 is shown in accordance with an exemplary embodiment. The needle platform 108 includes a cylindrical body 178, an upper circular extension 180 and a lower circular extension 182. The upper circular extension 180 and the lower circular extension 182 extend substantially horizontally from and perpendicular to the cylindrical body 178. As will be discussed in further detail herein, the upper circular extension 180 serves as a platform for the injection spring 112. The needle platform 108 further includes a first and second curved extensions 184. The curved extensions 184 extend substantially horizontally from and perpendicular to the cylindrical body 178 and extend substantially vertically between the upper circular extension 180 and the lower circular extension 182. Each curved extension 184 includes an angled surface 186. As will be discussed in further detail herein, when the needle platform is in the undeployed position, the angled surfaces 186 contact the hooks 156 of the inner sleeve 106, which prevents the needle platform 108 from transitioning to the deployed position.

The needle platform 108 further includes first and second projections 188. The first and second projections 188 are generally rectangular in shape, extend substantially horizontally from and perpendicular to the cylindrical body 178, and extend substantially vertically between the upper circular extension 180 and the lower circular extension 182. When the needle platform 108 is disposed within the inner sleeve 106, the projections 188 extend into the grooves 164 which guides the vertical movement of the needle platform 108, i.e., in a direction substantially parallel to the longitudinal axis of the lancet, while preventing a rotational or horizontal movement of the needle platform 108.

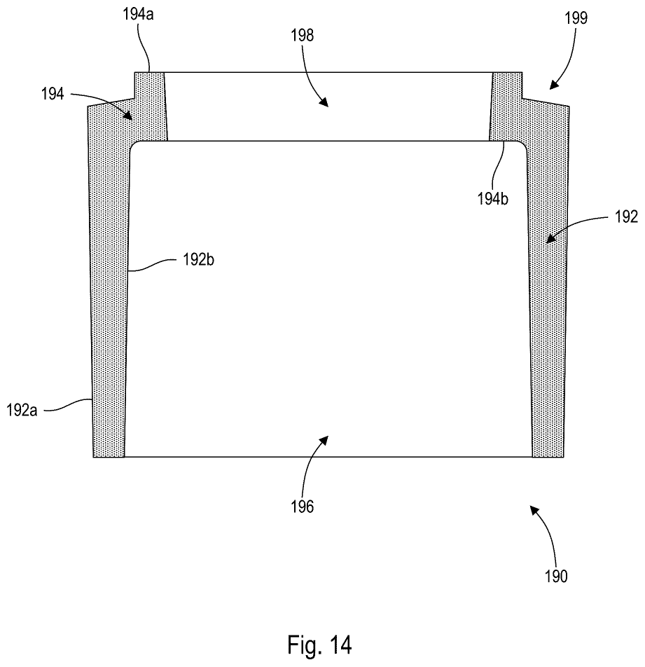

As depicted in FIG. 3, the lancet 100 further includes a spring sleeve 190. With particular reference to FIG. 14, the spring sleeve 190 is shown in accordance with an exemplary embodiment. The spring sleeve 190 is cylindrical in shape and includes a side wall 192 and a top wall 194. The side wall 192 includes an outer surface 192a and an opposed inner surface 192b and the top wall 194 includes a top surface 194a and an opposed bottom surface 194b. The inner surface 192b of the side wall 192 and the bottom surface 194b of the top wall 194 define an inner volume 196 of the spring sleeve 190. The side wall 192 and the top wall 194 are shaped and dimensioned such that the spring sleeve 190 retains the retraction spring 114 within the inner volume 196. The top wall 194 includes an opening 198 that extends therethrough. That is, the opening 198 extends between the top surface 194a and the bottom surface 194b of the top wall 194. The opening 198 of the top wall 194 is in communication with the inner volume 196. Since the inner volume 196 is open, the needles 110 may extend through spring sleeve 190 via the opening 198. The outer surface 192a defines a ledge 199 that extends circumferentially around the spring sleeve 190. As will be discussed in further detail herein, when the lancet 100 is in the undeployed position, the tabs 158 contact the ledge 199.

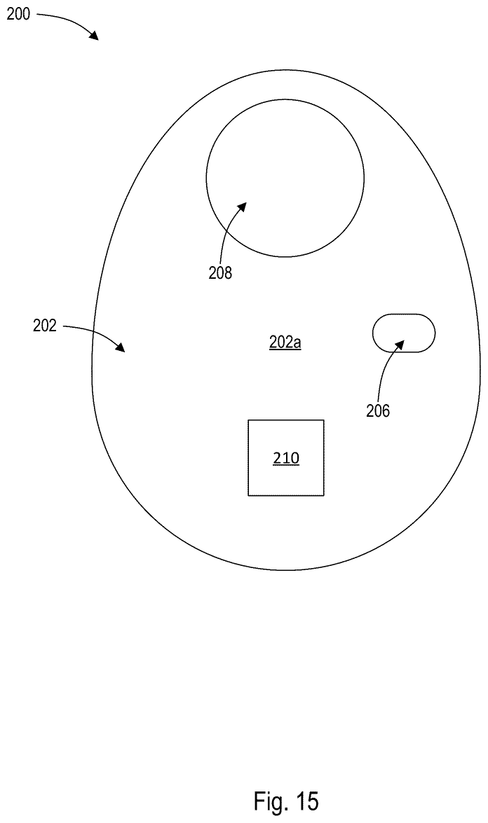

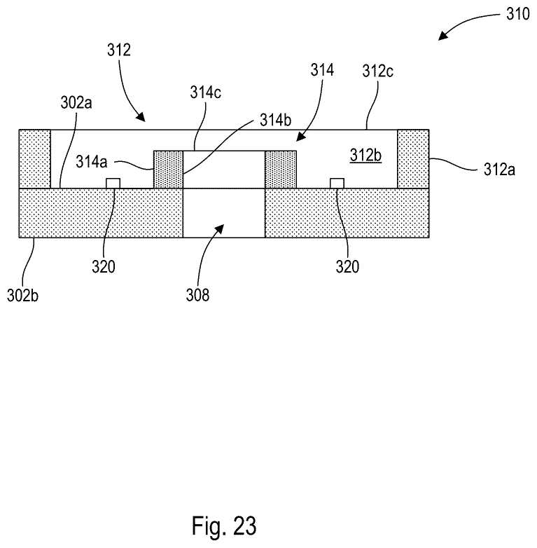

Referring now to FIGS. 15-25, the cartridge 12 is shown in accordance with an exemplary embodiment. The cartridge 12 is configured to attach to the skin of a subject via an adhesive layer (not shown) disposed on a bottom surface of the cartridge 12. After use, the cartridge 12 may be removed by pulling it off the skin of the subject. The cartridge 12 includes a cover 200 and a base 300 that can couple to the cover 200. In one embodiment, the cover 200 and the base 300 are formed as two separate components that are removably coupled to one another (e.g., via a snap fitting). In another embodiment, the cover 200 and the base 300 form an integral unitary cartridge 12. The cover 200 and the base 300 may be coupled to one another via an adhesive, laser welding, etc.

The cartridge 12 may be formed using a variety of suitable materials including, but not limited to, polymeric materials (e.g., polyolefins, polyethylene terephthalate (PET), polyurethanes, polynorbornenes, polyethers, polyacrylates, polyamides (Polyether block amide also referred to as Pebax®), polysiloxanes, polyether amides, polyether esters, trans-polyisoprenes, polymethyl methacrylates (PMMA), cross-linked trans-polyoctylenes, cross-linked polyethylenes, cross-linked polyisoprenes, cross-linked polycyclooctenes, inorganic-organic hybrid polymers, co-polymer blends with polyethylene and Kraton®, styrene-butadiene co-polymers, urethane-butadiene co-polymers, polycaprolactone or oligo caprolactone co-polymers, polylactic acid (PLLA) or polylactide (PL/DLA) co-polymers, PLLA-polyglycolic acid (PGA) co-polymers, photocross linkable polymers, etc.). In some embodiments, some of the cover 200 may be formed of poly(dimethylsiloxane) (PDMS) to allow visibility of components disposed within the cartridge 12.

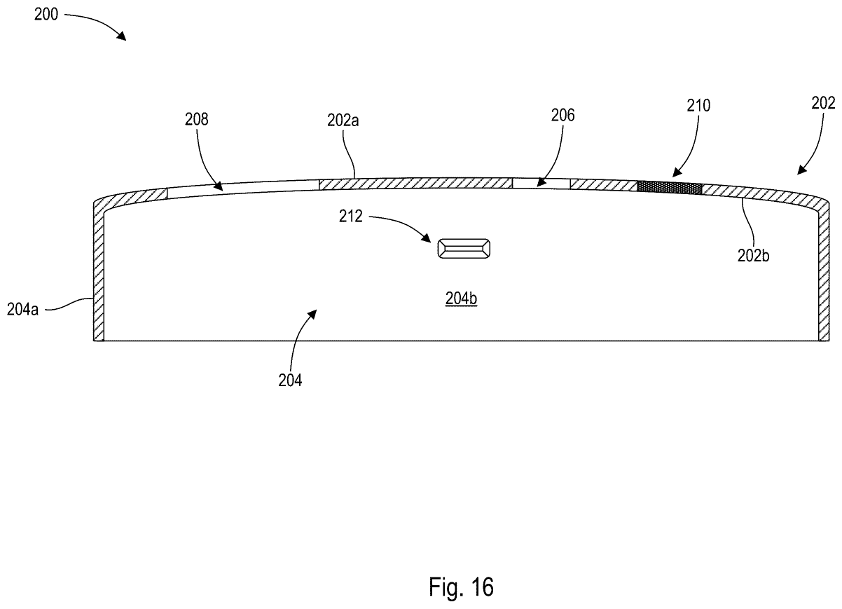

Referring now to FIGS. 15 and 16, the cover 200 is shown in accordance with an exemplary embodiment. The cover 200 includes a top wall 202 with an outer surface 202a and an opposed inner surface 202b. The cover 200 also includes a side wall 204 with an outer surface 204a and an opposed inner surface 204b. The top wall 202 extends substantially longitudinally from and perpendicular to the side wall 204. The side wall 204 extends substantially vertically from and perpendicular to the top wall 202.

The cover 200 includes a viewing aperture 206 that extends through the top wall 202. That is, the viewing aperture 206 extends between the outer surface 202a and the inner surface 202b of the top wall 202. In various embodiments, the viewing aperture 206 is covered by a transparent material, e.g., a transparent polymeric material, which allows viewing the components disposed within the cartridge 12. The top wall 202 further includes a lancet aperture 208 that extends through the top wall 202. That is, the lancet aperture 208 extends between the outer surface 202a and the inner surface 202b of the top wall 202. The lancet aperture 208 is generally shaped and dimensioned to accept a portion of the lancet 100, e.g., the aperture can be substantially circular. As will be discussed in further detail herein, a portion of the lancet 100 extends through the lancet aperture 208 to couple to the base 300.

The cover 200 also includes a biometric sensor (e.g., a fingerprint scanner, iris scanner, etc.) 210 that is configured to obtain a fingerprint of a user of the dermal patch system 10. While the biometric sensor 210 is disposed on the top wall 202 of the cover 200, in other embodiments, the biometric sensor 210 may be located elsewhere (e.g., on the side wall 204). The cover 200 further includes a plurality of locking members 212 that extend substantially horizontally from and perpendicular to the inner surface 204a of the side wall 204. As will be discussed in further detail herein, the locking members 212 aid in coupling the cover 200 to the base 300. For the biometric sensor, a finger image sensor (FIS), or a contact image sensor (CIS), such as the M116-A8CIU sensor or the M116-A6CIP sensor made by CMOS Sensor Inc. in Cupertino, CA, can be used. Also, optical finger image sensors can be used as they can be more sensitive and durable than capacitive fingerprint sensors, e.g., 4×6 mm sensing area. Optical finger image sensors generally include a light source and an optically clear cover, which is typically glass but a polymer or epoxy as substitute can be used. Alternatively, a capacitive fingerprint sensor that relies on the valleys and ridges of the finger could also be used. Also, flexible organic photodetectors can be used, as described in “Advances in Flexible Organic Photodetectors: Materials and Applications,” Nanomaterials 2022, 12, 3775 (https://doi.org/10.3390/nano12213775), which is hereby incorporated by reference in its entirety. For further information on organic photodetectors (OPD's), please also sec which also is hereby incorporated by reference in its entirety.

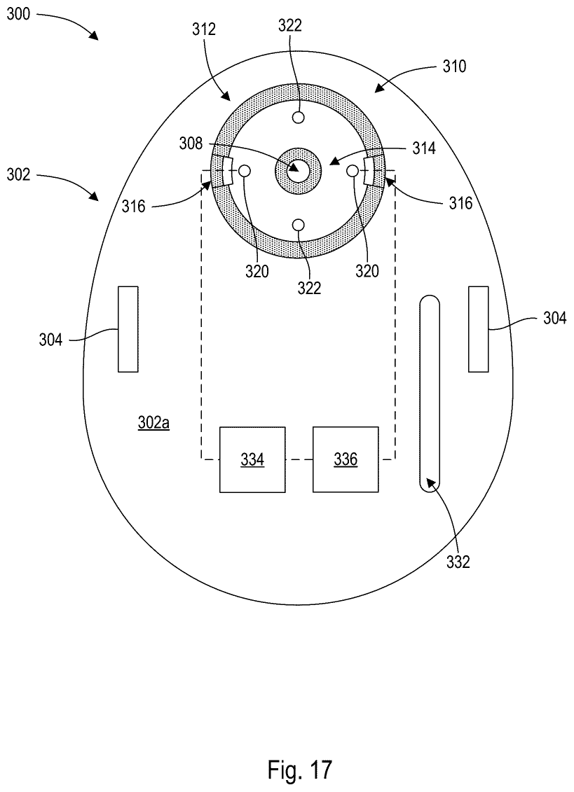

Referring now to FIGS. 17-25, the base 300 is shown in accordance with an exemplary embodiment. The base 300 includes a bottom wall 302 having a top surface 302a and an opposed bottom surface 302b. The bottom wall 302 has a similar shape and dimension as the side wall 204 of the cover 200 such that the cover 200 and the base 300 are flush with one another when the cover 200 is coupled to the base 300. Furthermore, when the cover 200 is coupled to the base 300, the side wall 204 contacts the bottom wall 302.

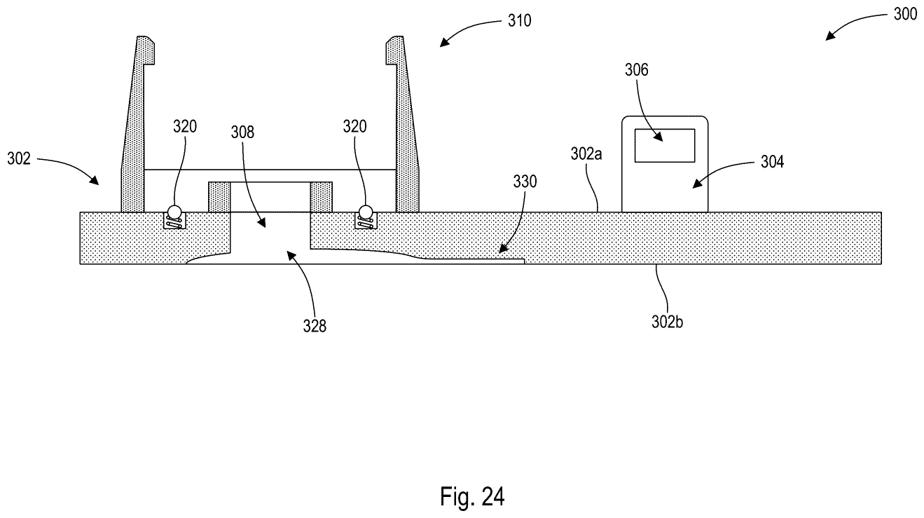

The base 300 includes a plurality of extensions 304 that extend substantially vertically from and perpendicular to the top surface 302a of the bottom wall 302. Each of the extensions 304 define a gap 306 (FIG. 24). The gap 306, and therefore the extensions 304, are shaped and dimensioned to accept a locking member 212. The locking members 212 extend through the gaps 306 to couple the cover 200 to the base 300.

The base 300 further includes a needle aperture 308 that is generally circular in shape. The needle aperture 308 extends through the bottom wall 302. That is, the needle aperture 308 extends between the top surface 302a and the bottom surface 302b of the bottom wall 302. As will be discussed in further detail herein, when the cover 200 is coupled to the base 300 and when the cartridge 12 is adhered to a subject, the needle aperture 308 allows the needles 110 of the lancet 100 to extend through the bottom wall 302 to puncture the subject's skin.

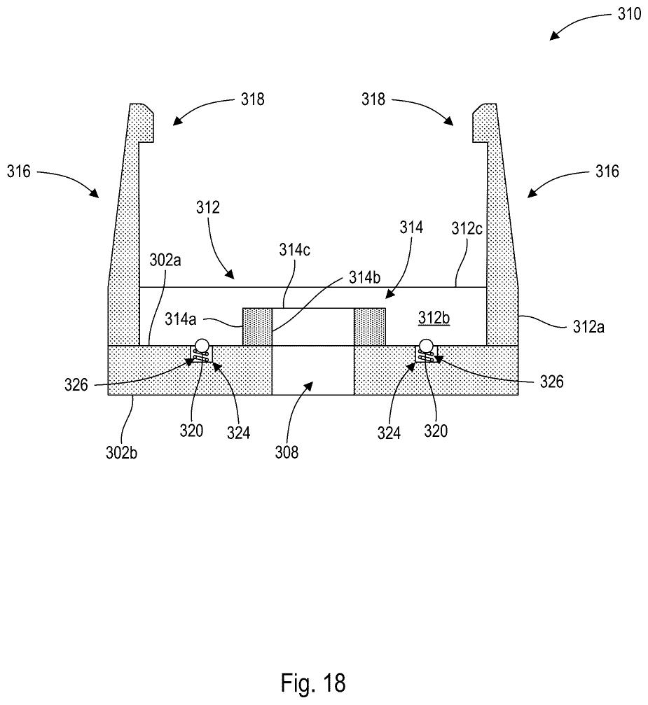

The base 300 also includes lancet receiving element 310 that is shaped and dimensioned to accept a distal end of the lancet 100. With particular reference to FIGS. 18 and 20-23, the lancet receiving element 310 includes an outer circular projection 312 and an inner circular projection 314, each extending substantially vertically from and perpendicular to the top surface 302a of the bottom wall 302. The outer circular projection 312 includes an outer surface 312a, an opposed inner surface 312b, and a top surface 312c that extends between the outer surface 312a and the inner surface 312b. The top surface 312c extends between the outer surface 312a and the inner surface 312b and is substantially perpendicular to those surfaces. The outer surface 312a and the inner surface 312b extend substantially vertically from and perpendicular to the top surface 302a of the bottom wall 302. The outer circular projection 312 is shaped to accept the lancet 100.

The inner circular projection 314 is disposed around the needle aperture 308 and includes an outer surface 314a, an opposed inner surface 314b, and a top surface 314c that extends between the outer surface 314a and the inner surface 314b. The top surface 314c extends between the outer surface 314a and the inner surface 314b and is substantially perpendicular to those surfaces. The outer surface 314a and the inner surface 314b extend substantially vertically from and perpendicular to the top surface 302a of the bottom wall 302. The outer circular projection 312 and the inner circular projection 314 circular projection are substantially concentric with one another.

As will be discussed in further detail herein, when the lancet 100 is engaged with the cartridge 12, the top surface 314c of the inner circular projection contacts a portion of the lancet 100 which causes the lancet 100 to transition the needles 110 from the undeployed position to the deployed position.

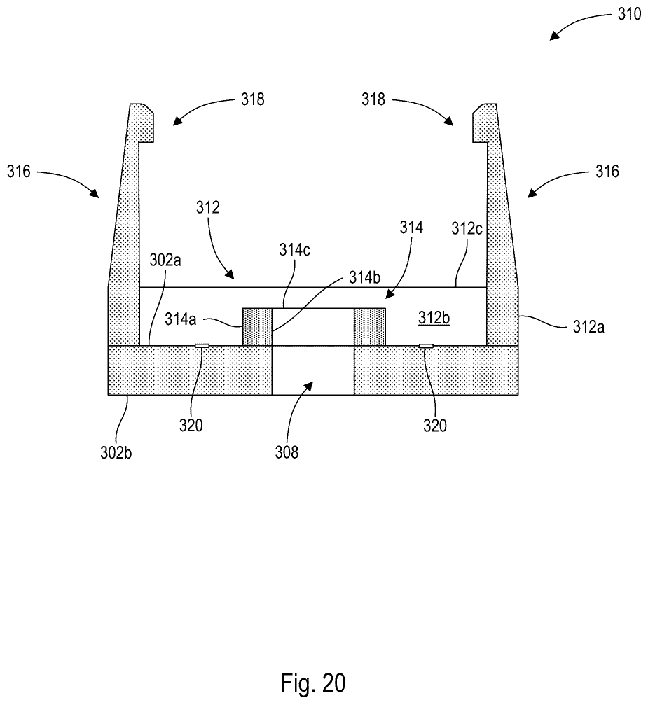

Withe continued reference to FIGS. 18 and 20-22, the lancet receiving element 310 further includes a plurality of locking members 316 that extend substantially vertically from and perpendicular to the top surface 312c of the outer circular projection 312. While FIGS. 20-22 depict the lancet receiving element 310 as including two locking members 316, it is understood that, in other embodiments, the lancet receiving element 310 may include more or less locking members 316 (e.g., 1, 3, 5, etc.). Each locking member 316 includes a hook 318 that extends inwardly from a top of a locking member 316 towards the inner circular projection 314. As will be discussed in further detail herein, the hooks 318 of the locking members 316 couple to the lancet 100 to retain the lancet 100 within the base 300.

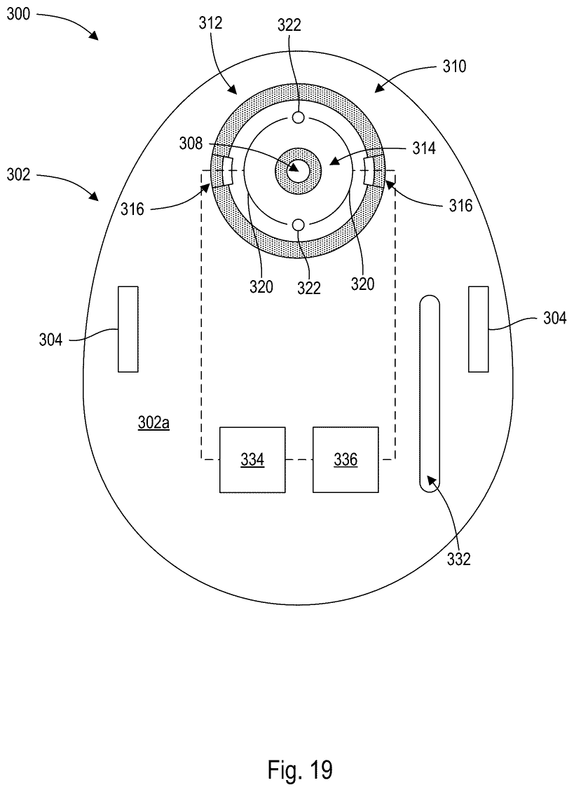

The lancet receiving element 310 further includes a plurality of electrical contacts 320 and a plurality of extensions 322. The extensions 322 are substantially cylindrical in shape and extend substantially from and perpendicular to the top surface 302a of the bottom wall 302. The electrical contacts 320 are in electrical communication with one another and are formed of a material that is suitable for conducting electricity (e.g., copper, iron, gold, aluminum, etc.). In the embodiment depicted in FIGS. 17 and 18, the electrical contacts 320 are in the form of pogo style connectors. In this embodiment, the bottom wall 302 includes contact receptacles 324 each of which houses one of the electrical contacts 320 and a respective spring 326. When the lancet 100 is not engaged with the cartridge 12 the springs 326 are in a relaxed state and push the contacts 320 partially beyond the bottom wall 302. In another embodiment (FIGS. 19 and 20), the electrical contacts 320 may take the form of an electrically conductive pad that extends substantially vertically from and perpendicular to the top surface 302a of the bottom wall 302. In these embodiments, the contact receptacles 324 and the springs 326 are omitted.

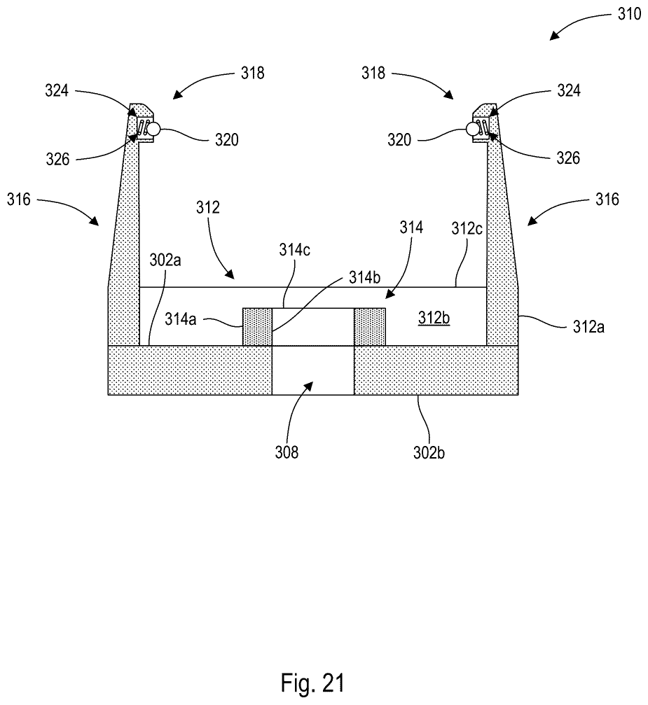

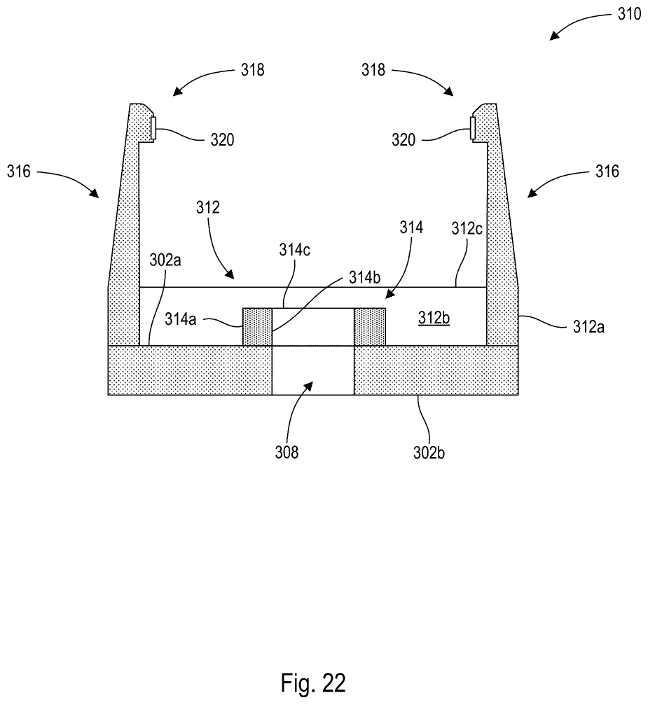

While FIGS. 17-20, depict the electrical contacts 320 as located on the bottom wall 302, in another embodiment, when electrical contacts 132 of the lancet 100 are disposed on the side wall 116, the hooks 318 of the locking members 316 can include the electrical contacts 320. In one embodiment (FIG. 21), where the electrical contacts 320 are in the form of pogo style connectors, the hooks 318 can include the contact receptacles 324 and the springs 326. In another embodiment (FIG. 22), the electrical contacts 320 disposed on the hooks 318 can take the form of an electrical pad and, in this embodiment, the contact receptacles 324 and the springs 326 are omitted.

With particular reference to FIG. 24, the base 300 includes a physiological sample well 328 and a physiological sample channel 330 that extends radially from and is in open communication with the physiological sample well 328. As will be discussed in further detail herein, the physiological sample channel 330 is a fluidic channel that is configured to carry a physiological sample extracted from a subject. The physiological sample well 328 and a portion of the physiological sample channel 330 are open with respect to the bottom surface 302b of the bottom wall 302. Stated another way, the physiological sample well 328 and a portion of the physiological sample channel 330 do not include a bottom surface. The physiological sample well 328 is in open communication with the needle aperture 308. As will be discussed in further detail herein, when drawing a physiological sample, the needles 110 of the lancet 100 extend through the needle aperture 310 and through the physiological sample well 328 to pierce the skin of the subject. The physiological sample channel 330 extends from the physiological sample well 328 and through the bottom wall 302 of the base 300.

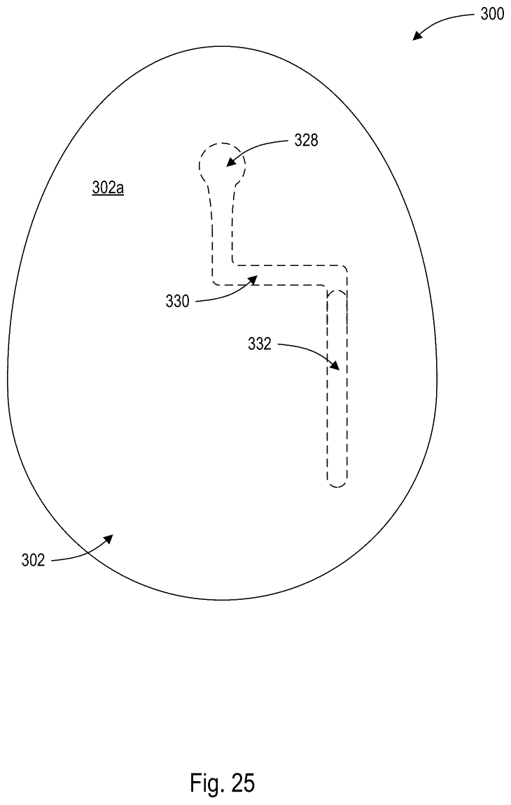

The bottom wall 302 of the base 300 further includes a sample collection reservoir 332 (FIG. 25) that is configured to receive and store a drawn physiological sample. The sample collection reservoir 332 is in open communication with the physiological sample channel 330. Accordingly, the physiological sample channel 330 carries a drawn physiological sample from the physiological sample well 328 to the sample collection reservoir 332 via capillary action. The sample collection reservoir 332 is disposed below the viewing aperture 206. In one embodiment, the collection reservoir 332 is covered by a transparent material which allows a user of the dermal patch system 10 to view the sample collection reservoir 332 via the viewing aperture 206. In some embodiments, a sample collection pad (e.g., a CF12 collection pad) is disposed within the sample collection reservoir 332 and absorbs the drawn physiological sample.

The base 300 includes an adhesive layer (not shown) disposed on the bottom surface 302b of the bottom wall 302. The cartridge 12 can be attached to the skin of a subject via the adhesive layer. The cartridge 12 may be attached anywhere on the subject's skin that is capable of supporting the cartridge 12 (e.g., on a leg, arm, etc. of the subject). The adhesive layer includes an opening that surrounds the physiological sample well 328 and through which the needles 110 can extend to puncture the subject's skin. The adhesive layer covers and therefore seals the open portion of the physiological sample channel.

The base 300 further includes a computer system (e.g., a SOC) 334 and a power supply (e.g., a battery, charged capacitor, etc.) 336. The computer system 334 is connected to and in communication with the biometric sensor 210. The power supply 336 is in electrical communication with the biometric sensor 210, the computer system 334, and the electrical contacts 320 such that in absence of engagement of the lancet with the cartridge, an open circuit is established that prevents the application of power by the power supply 336 to the biometric sensor 210 and the computer system 334. As will be discussed in further detail herein, when the lancet 100 is engaged with the cartridge 12 the circuit is closed which allows the power supply 336 to power the biometric sensor 210 and the computer system 334. As will be discussed in further detail herein, when powered, the biometric sensor 210 obtains biometric data, such as fingerprint data, from a user of the dermal patch system 10.

Before the lancet 100 engages with the cartridge 12, the needles 110 are in an undeployed position (FIG. 3). In the undeployed position, the hooks 156 of the inner sleeve 106 extend through the openings 126 of the housing 102, which retains the lancet 100 in the undeployed position and prevents the lancet 100 from moving to the deployed position. In this position, the extensions 152 are compressed inward toward the center of the lancet 100, which allows the needle platform 108 to rest upon the inner sleeve 106 thereby preventing the needle platform from moving vertically downward and into the deployed position. Specifically, the angled surface 186 of the extensions 184 of the needle platform 108 rests upon the extensions 152, thereby inhibiting the movement of the needle platform.

Furthermore, the injection spring 112 extends around the body 178 of the needle platform 108 and the outer surface 144a of the inner cylinder 144. The injection spring 112 extends substantially vertically between the inner surface 140b of the top wall 140 of the cap 104 and the upper circular extension 180 of the needle platform 108. In the undeployed position, the injection spring 112 is compressed between the top wall 140 of the cap 104 and the upper circular extension 180 of the needle platform 108. The retraction spring 114 extends around the post 168 of the inner sleeve 106. The retraction spring 114 extends substantially vertically between the bottom wall 148 of the inner sleeve 106 and the top wall 194 of the spring sleeve 190. That is, the retraction spring 114 extends substantially vertically between the inner surface 148b of the bottom wall 148 of the inner sleeve 106 and the inner surface 194b of the top wall 194 of the spring sleeve 190. When the lancet 100 is in the undeployed position, the retraction spring 114 is compressed between the bottom wall 148 of the inner sleeve 106 and the top wall 194 of the spring sleeve 190. Furthermore, in the undeployed position, the deformable tabs 158 contact the ledge 199 of the spring sleeve 190 so as to prevent the retraction spring from expanding and moving the spring sleeve 190.

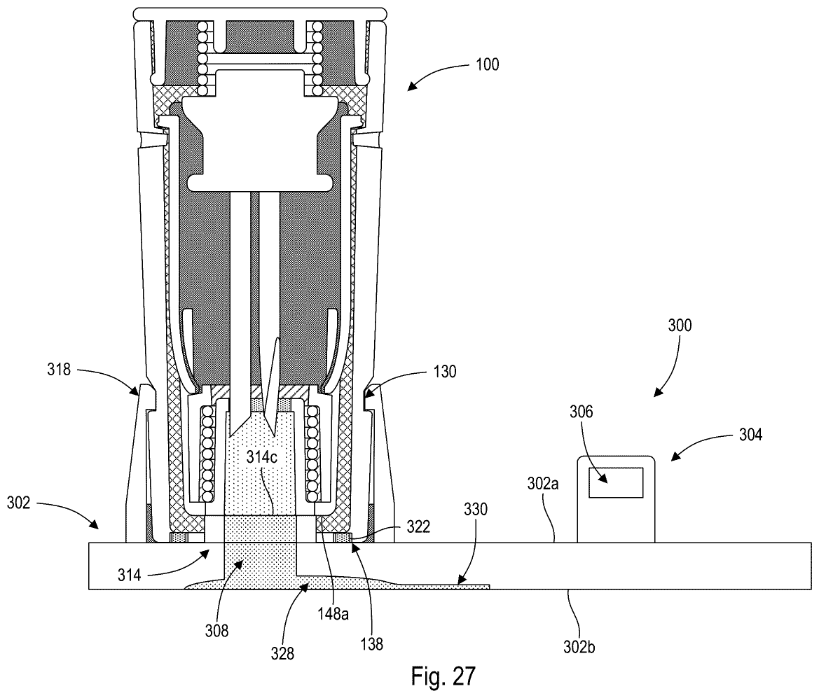

The lancet 100 can be engaged with the cartridge 12 by pushing the lancet 100 into the cartridge 12. In order to transition the needles 110 to the deployed position and power the biometric sensor 210 and the computer system 334, the lancet 100 must be properly aligned with the cartridge 12 before engagement. When the lancet 100 is not properly aligned with the cartridge 12, the extensions 304 contact the outer surface 148a of the bottom wall 148 which prevents the lancet 100 from deploying the needles 110. When the lancet 100 is properly aligned with the cartridge 12, the extensions 304 extend into the openings 138 (FIG. 27). In this position the electrical contacts 132 and the electrical contacts 320 become physically engaged, e.g., they touch one another, and the lancet 100 is able to deploy the needles 110 to draw a physiological sample from a subject.

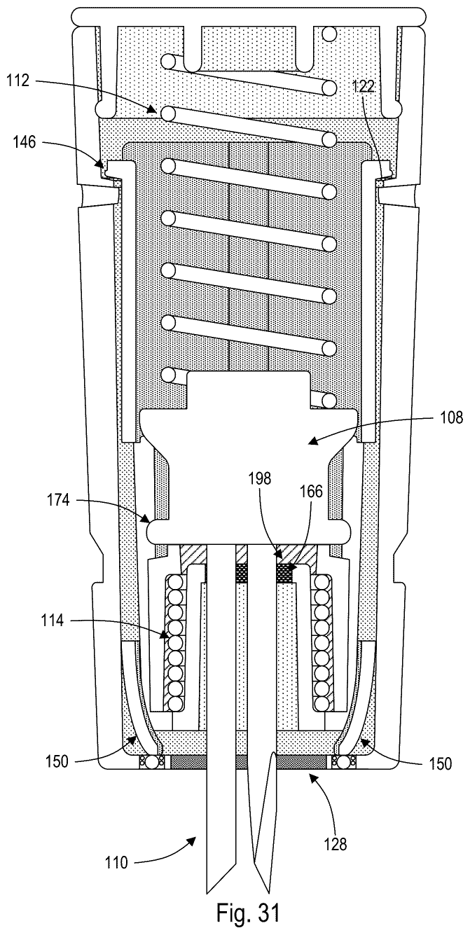

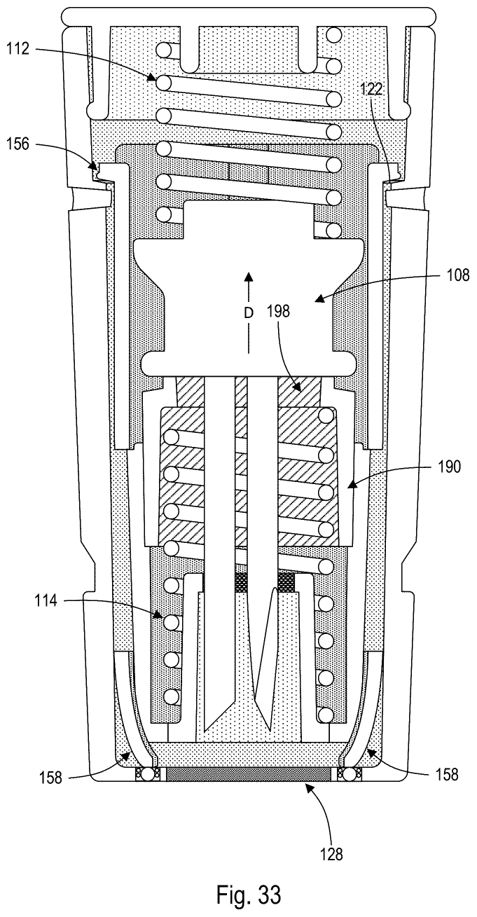

The engagement of the lancet 100 with the cartridge 12 causes the needles 110 of the lancet 100 to automatically move from an undeployed position (FIG. 3) to a deployed position (FIG. 31) and automatically from the deployed position to a retracted position (FIG. 33). Furthermore, when the lancet 100 engages with the base 300, the hooks 318 of the locking members 316 couple to the groove 130 of the housing 102 via a snap fitting. This coupling causes the generation of an audible clicking sound that indicates to a user that the lancet 100 was correctly inserted into the cartridge 12.

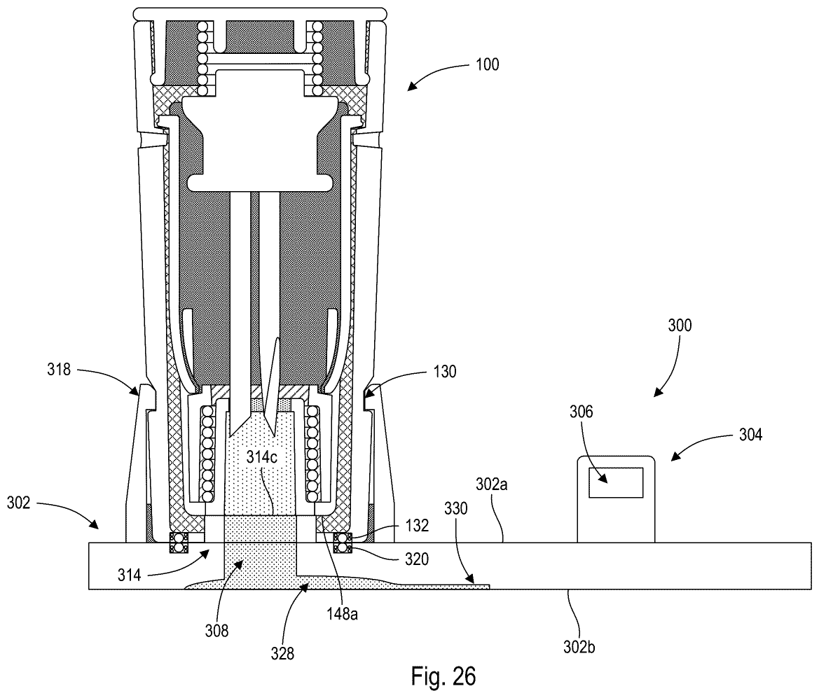

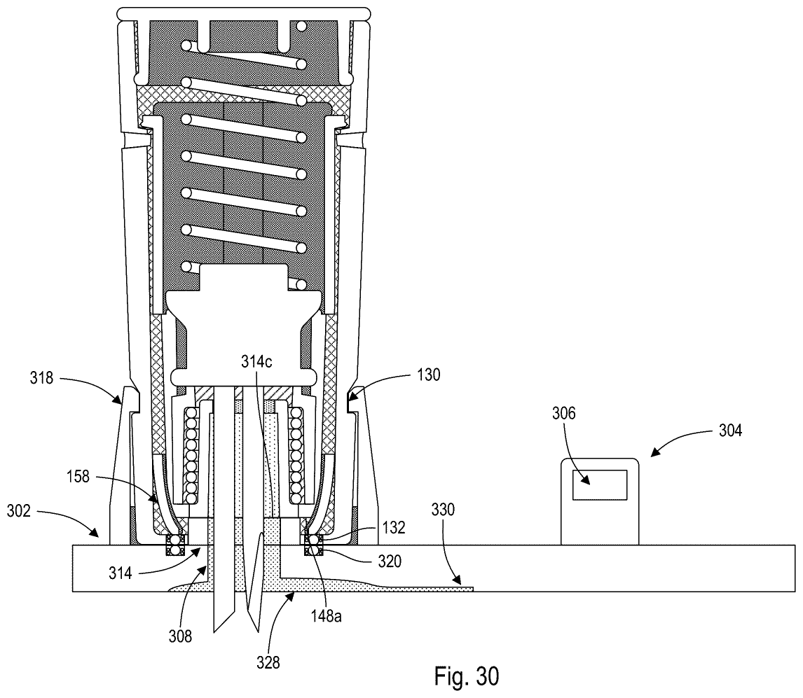

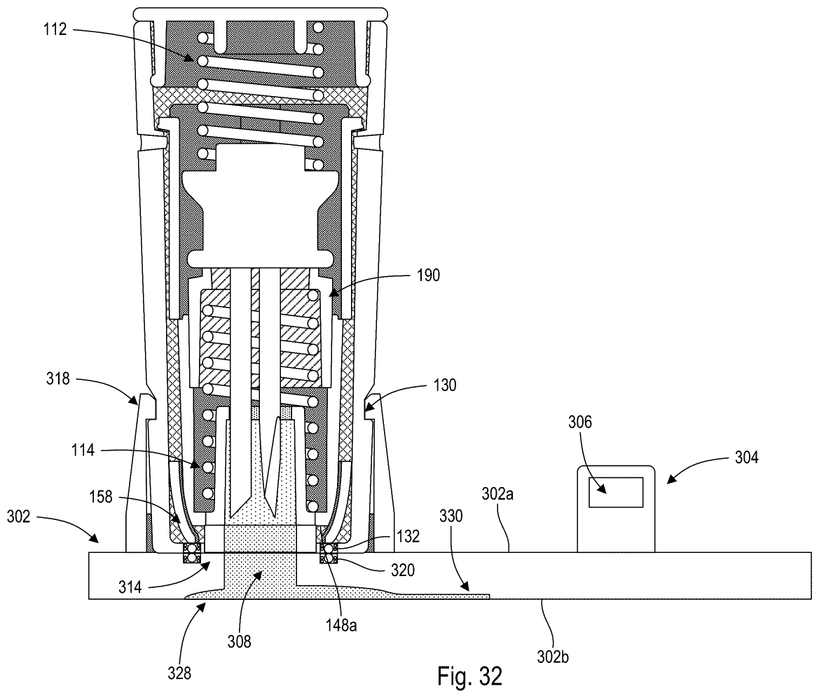

Upon engagement with the cartridge 12, the electrical contacts 132 and the springs 136 are compressed into the contact receptacles 134 by the electrical contacts 320. When the electrical contacts 320 are also pogo style connectors, the electrical contacts 320 and the springs 326 are also compressed into the contact receptacles 324. While FIGS. 26, 30, and 32 depict the electrical contacts 320 as pogo style connectors, it is understood that the electrical contacts 320 may be electrically conductive pads as depicted in FIGS. 19 and 20. Furthermore, while FIGS. 21 and 22 depict the bottom wall 118 as including the electrical contacts 132 in other embodiments, as depicted in FIGS. 21 and 22, the side wall 116 can include the electrical contacts 132. In some such embodiments, the hooks 318 include the electrical contacts 320.

When the lancet 100 is coupled to the base 300 the inner circular projection 314 of the lancet receiving element 310 extends through the aperture 128 of the housing 102 and contacts the bottom wall 148 of the inner sleeve 106. Specifically, the top surface 314c of the inner circular projection 314 contacts the outer surface 148a of bottom wall 148 which forces the inner sleeve 106 to move vertically upward in the direction of arrow A within the housing 102. As previously discussed herein, grooves 124 of the housing 102 and the protrusions 162 of the inner sleeve 106 guide the vertical movement of the inner sleeve 106. The vertical movement of the inner sleeve 106 causes the hooks 156 to disengage from the openings 126 and causes the extensions 152 to extend vertically above and rest upon the ledge 122. In this position, the extensions 152 decompress and expand outwardly in the direction of arrow B.

When the extensions 152 expand, the needle platform 108 no longer rests upon the extensions 152 and hence can move vertically downward in the direction of arrow C. As the needle platform 108 moves downward, the injection spring 112 is allowed to expand and the force applied by the injection spring 112 causes the needle platform 108 (and therefore the needles 110) to travel at a rate that is sufficient to cause the needles 110 to puncture the skin of a subject wearing the dermal patch system 10. The expansion of the injection spring 112 causes the needles 110 to move and extend through the spring sleeve 190 via the opening 198, through the post 168 via the opening 174, through the opening 166 of the inner sleeve 106 and exit the housing 102 via the aperture 128 and pass through the base 300 of the cartridge 12 via the needle aperture 308. Stated another way, the injection spring 112 moves the needles 110 to the deployed position.

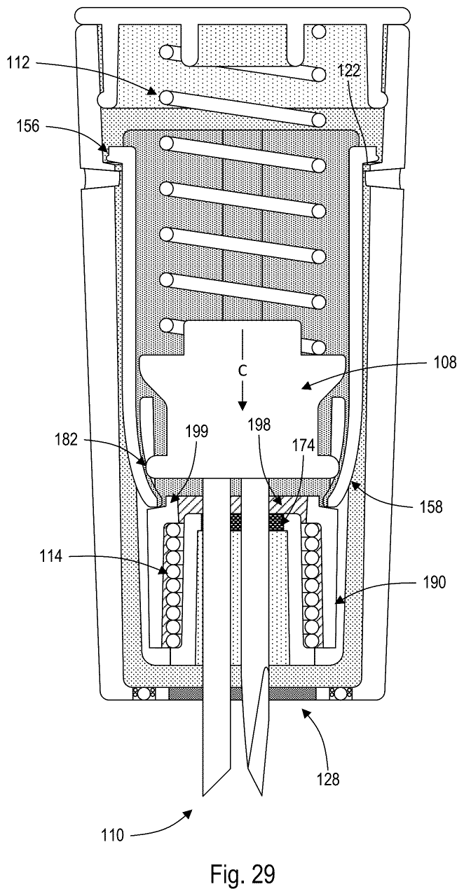

As previously discussed herein, the grooves 164 of the inner sleeve 106 and the projections 188 of the needle platform 108 guide the vertical movement of the needle platform 108. As the needle platform moves in the direction of arrow B, the lower circular extension 182 contacts the deformable tabs 158 (FIG. 29). This contact causes the tabs 158 to deflect away from the spring sleeve 190 of the lancet 100 or causes the tabs 158 to break. As depicted in FIG. 31, when the tabs 158 break, the broken piece(s) of the tabs 158 fall into a void (receptacle) between the inner sleeve 106 and the housing 102. In the deployed position, the needles 110 extend beyond the housing 102 and the lower circular extension 182 contacts the top wall 194 of the spring sleeve 190.

When the tabs 158 deflect away from the spring sleeve 190 or break, the tabs 158 no longer contact the ledge 199 of the spring sleeve 190. As a result, the retraction spring 114 is allowed to expand. The expansion of the retraction spring 114 moves the spring sleeve 190 vertically upward in the direction of arrow D. If the tabs 158 deflect and do not break, the spring sleeve 190 prevents the tabs 158 from contacting the retraction spring 114 as the retraction spring 114 expands. The spring sleeve 190 moves the needle platform 108 in the direction of arrow D to place the lancet 100 in the retracted position. That is, after moving to the deployed position, the retraction spring 114 causes the needles 110 to retract back into the inner volume 150 of the inner sleeve 106 via the needle aperture 308 of the base 300, the aperture 128 of the housing 102, and the opening 166 of the inner sleeve 106. That is, after penetrating the skin of a subject, the retraction spring 114 causes the needles 110 to automatically retract back into the housing of the lancet 100 thereby placing the lancet 100 in the retracted position.

In this embodiment, the three needles 110 have substantially the same length, and by simultaneously moving the three needles 110 to the deployed position, the lancet 100 punctures the skin of the subject at three locations at the same time, though in other embodiments, two or all of the three needles 110 may have different lengths.

In some embodiments, the use of three needles 110 results in placing a portion of the skin under tension, which can in turn facilitate drawing blood through on or more puncture sites caused by the needles. In particular, as noted above, it has been discovered that the use of three needles 110 can advantageously allow a larger volume of blood to be drawn compared to the use of one, two, or a greater number of needles 110. Without being limited to any particular theory, in some embodiments, in addition to placing a skin portion under tension, the use of three needles 110 may also improve the likelihood that at least one of the needles would cut through a capillary to extract a blood volume from the subject. Each of the needles 110 has a tapered tip that points outward (away from the center of the lancet) to maximize a distance between puncture locations on the subject's skin. Furthermore, the three needles 110 may be positioned such that the needle tips form an equilateral triangle and are therefore equally space from one another.

It is understood that the lancet 100 may be used with any cartridge of a dermal patch system that is configured to engage with the lancet 100. That is, the lancet 100 may be used with any cartridge that includes an extension that causes the lancet 100 to transition from the undeployed position to the deployed position.

After the needles 110 retract, a physiological sample pools within the physiological sample well 328 of the base 300. Due to gravity, wicking, and capillary action, the physiological sample travels from the physical sample well 328 to the sample collection reservoir 332 via the physiological sample channel 330. A user can view the sample collection reservoir 332 via the viewing aperture 206 to ensure the physiological sample has traveled to the sample collection reservoir 332. After drawing the physiological sample, the cartridge 12 may be removed from the skin of the subject and sent to a laboratory for analysis. A medical professional may then extract the physiological sample and analyze the sample.

As previously discussed herein, when the lancet 100 is properly engaged with the cartridge 12, the electrical contacts 132 engage the electrical contacts 320. When the electrical contacts 132 and the electrical contacts 320 are engaged, a closed electrical circuit is established between the biometric sensor 210, the computer system 334 and the power supply 336. In one embodiment, the cover 200 can include a power button (not shown) that when the lancet 100 is engaged with the cartridge 12 and the power button is pushed, the power supply 336 powers the biometric sensor 210 and the computer system 334. In another embodiment, the power supply 336 is a charged capacitor, when the circuit is closed, the power supply 336 automatically powers the biometric sensor 210 and the computer system 334.

In embodiments in which the biometric sensor 210 is a fingerprint scanner, while a user obtains the physiological sample from a subject, the user or subject can place their finger on the fingerprint scanner 210 such that the user's or subject's fingerprint data can be collected. When powered, the biometric sensor 210 obtains the user's or subject's fingerprint data and sends the fingerprint data to the computer system 334, where it can be stored in a memory module of the computer system 334. The biometric sensor 210 also sends the date and the time that the biometric data, in this embodiment fingerprint data, to the computer system 334. The stored fingerprint data, the date, and the time can be used to establish a chain of custody of the cartridge 12 and therefore the drawn physiological sample. That is, the stored fingerprint data can be matched to a fingerprint of the user or the subject at a later time to establish that the subject was subject was the individual from whom the physiological sample was drawn or the user was the individual that drew the physiological sample from the subject. For example, the memory of the computer system may be accessed to obtain the stored fingerprint data and comparted to other fingerprint data that was obtained before or after the fingerprint data stored in the memory of the computer system. This data may be compared using various processing systems.

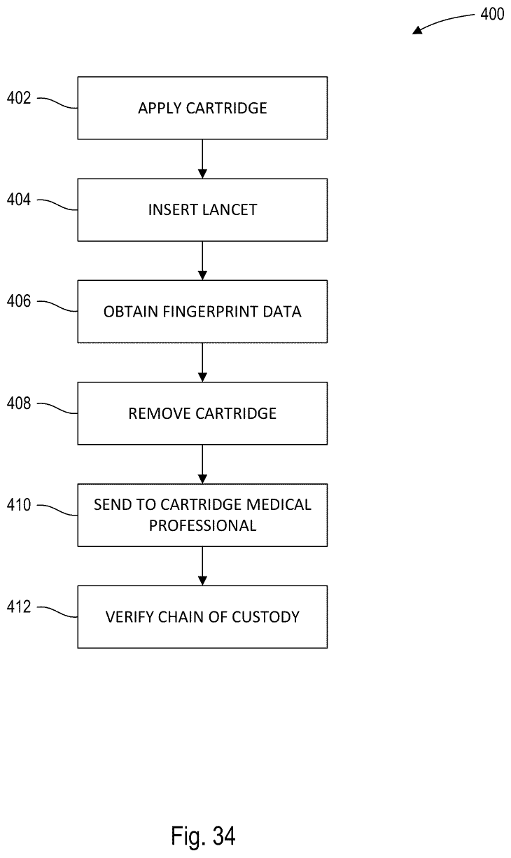

Referring now to FIG. 34, a method 400 for obtaining a physiological sample from a subject is disclosed herein.

At 402, a user attaches the cartridge 12 of the dermal patch system 10 to the skin of a subject at a suitable location (e.g., arm, leg, etc.) as previously discussed herein.

At 404, the lancet 100 engages with the cartridge 12 to draw the physiological sample and allow the power supply 336 to power the biometric sensor 210 and the computer system 334 as previously discussed herein.

At 406, the biometric sensor 210 obtains fingerprint data from the user of the dermal patch system 10 and sends a signal indicative of the fingerprint data to the computer system 334 as previously discussed herein. In response to receiving the signal indicative of the fingerprint data, the computer system 334 stores the fingerprint data in a memory of the computer system 334.

At 408, the cartridge 12 is removed from the skin of the subject as previously discussed herein.

At 410, the cartridge 12 is sent to a medical professional to analyze the drawn physiological sample as previously discussed herein.

At 412, a chain of custody of the cartridge 12 is established by verifying an individual's fingerprint matches the fingerprint data stored in the memory of the computer system 334.

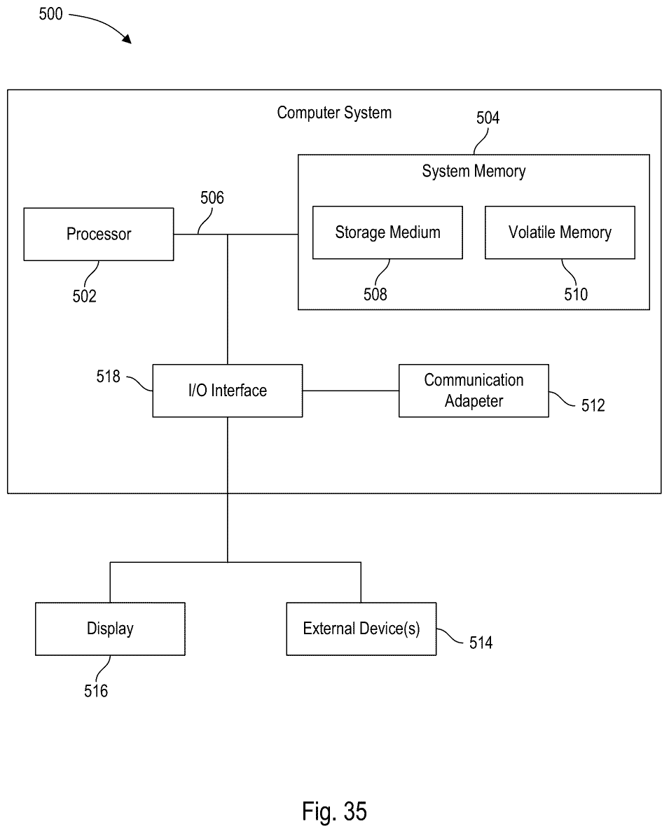

Referring now to FIG. 35, a computer system 500 is shown in accordance with an exemplary embodiment. The computer system 500 may serve as any computer system disclosed herein. As used herein a computer system (or device) is any system/device capable of receiving, processing, and/or sending data. Computer systems include, but are not limited to, microprocessor-based systems, a system on a chip (“SOC”) personal computers, servers, hand-held computing devices, tablets, smartphones, multiprocessor-based systems, mainframe computer systems, virtual reality (“VR”) headsets and the like.

As shown in FIG. 35, the computer system 500 includes one or more processors or processing units 502, a system memory 504, and a bus 506 that couples the various components of the computer system 500 including the system memory 504 to the processor 502. The system memory 504 includes a computer readable storage medium 508 and volatile memory 510 (e.g., Random Access Memory, cache, etc.). As used herein, a computer readable storage medium includes any media that is capable of storing computer readable; program instructions and is accessible by a processor. The computer readable storage medium 508 includes non-volatile and non-transitory storage media (e.g., flash memory, read only memory (ROM), hard disk drives, etc.). Computer program instructions as described herein include program modules (e.g., routines, programs, objects, components, logic, data structures, etc.) that are executable by a processor. Furthermore, computer readable program instructions, when executed by a processor, can direct a computer system to function in a particular manner such that a computer readable storage medium comprises an article of manufacture. Specifically, the computer readable program instructions when executed by a processor can create a means for carrying out at least a portion of the steps of the methods disclosed herein.

The bus 506 may be one or more of any type of bus structure capable of transmitting data between components of the computer system 500 (e.g., a memory bus, a memory controller, a peripheral bus, an accelerated graphics port, etc.).

The computer system 500 may further include a communication adapter 512 which allows the computer system 500 to communicate with one or more other computer systems/devices via one or more communication protocols (e.g., Wi-Fi, BTLE, etc.) and in some embodiments may allow the computer system 500 to communicate with one or more other computer systems/devices over one or more networks (e.g., a local area network (LAN), a wide area network (WAN), a public network (the Internet), etc.).

In some embodiments, the computer system 500 may be connected to one or more external devices 514 and a display 516. As used herein, an external device includes any device that allows a user to interact with a computer system (e.g., mouse, keyboard, touch screen, etc.). An external device 514 and the display 516 may be in communication with the processor 502 and the system memory 504 via an Input/Output (I/O) interface 518.

The display 516 may display a graphical user interface (GUI) that may include a plurality of selectable icons and/or editable fields. A user may use an external device 514 (e.g., a mouse) to select one or more icons and/or edit one or more editable fields. Selecting an icon and/or editing a field may cause the processor 502 to execute computer readable program instructions stored in the computer readable storage medium 508. In one example, a user may use an external device 514 to interact with the computer system 500 and cause the processor 502 to execute computer readable program instructions relating to at least a portion of the steps of the methods disclosed herein.

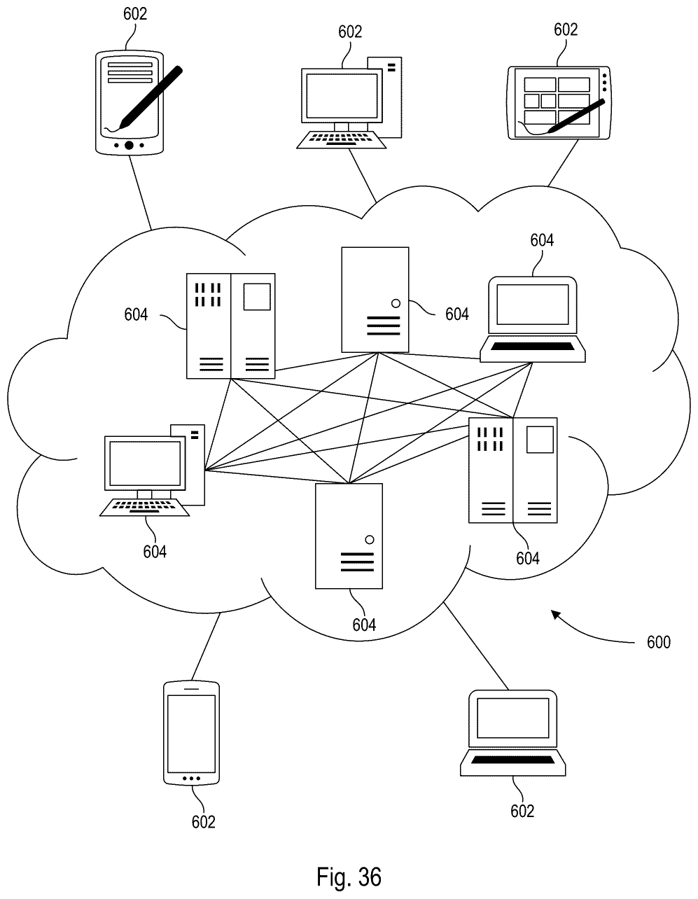

Referring now to FIG. 36, a cloud computing environment 600 is depicted in accordance with an exemplary embodiment. The cloud computing environment 600 is connected to one or more user computer systems 602 and provides access to shared computer resources (e.g., storage, memory, applications, virtual machines, etc.) to the user computer systems 602. As depicted in FIG. 36, the cloud computing environment includes one or more interconnected nodes 604. Each node 604 may be a computer system or device local processing and storage capabilities. The nodes 604 may be grouped and in communication with one another via one or more networks. This allows the cloud computing environment 600 to offer software services to the one or more computer services to the one or more user computer systems 602 and as such, a user computer system 602 does not need to maintain resources locally.

In one embodiment, a node 604 includes computer readable program instructions for carrying out various steps of various methods disclosed herein. In these embodiments, a user of a user computer system 602 that is connected to the cloud computing environment may cause a node 604 to execute the computer readable program instructions to carry out various steps of various methods disclosed herein.

As previously discussed, the above may be implemented by way of computer readable instructions, encoded or embedded on computer readable storage medium (which excludes transitory medium), which, when executed by a processor(s), cause the processor(s) to carry out the methods of the present disclosure.

While various embodiments have been illustrated and described in detail in the drawings and foregoing description, such illustration and description are to be considered illustrative or exemplary and not restrictive; embodiments of the present disclosure are not limited to the disclosed embodiments. Other variations to the disclosed embodiments can be understood and effected by those skilled in the art in practicing embodiments of the present disclosure, from a study of the drawings, the disclosure, and the appended claims.

In the claims, the word “comprising” does not exclude other elements or steps, and the indefinite article “a” or “an” does not exclude a plurality. A single processor or other processing unit may fulfill the functions of several items recited in the claims. The mere fact that certain measures are recited in mutually different dependent claims does not indicate that a combination of these measured cannot be used to advantage. Any reference signs in the claims should not be construed as limiting the scope.

What is claimed is:

1. A dermal patch system for collecting a physiological sample from a subject, comprising:
a cartridge configured to attach to the skin of the subject, and
a lancet configured to engage with the cartridge and draw a physiological sample upon engagement,
wherein the cartridge includes a biometric sensor configured to obtain biometric data from a subject.

2. The dermal patch system of claim 1, wherein the cartridge further includes:
a computer system configured to receive the biometric data from the and store the biometric data within a memory of the computer system.

3. The dermal patch system of claim 2, wherein the cartridge further includes:
a power supply configured to power the biometric sensor and the computer system.

4. The dermal patch system of claim 3, wherein the power supply is configured to power the biometric sensor and the computer system when the lancet engages with the cartridge.

5. The dermal patch system of claim 4, wherein the power supply is connected to the biometric sensor and the computer system such that a closed circuit is established only in response to the engagement of the lancet with the cartridge.

6. The dermal patch system of claim 5, wherein the cartridge includes first electrical contacts and the lancet includes second electrical contacts, wherein the engagement of the lancet with the cartridge results in engagement of the first and second electrical contacts so as to establish said closed circuit.

7. The dermal patch system of claim 6, wherein the first and second electrical contacts are pogo style connectors.

8. The dermal patch system of claim 6, wherein the first electrical contacts are electrically conductive pads and the second electrical contacts are pogo style connectors

9. The dermal patch system of claim 6, wherein the lancet includes a needle and wherein the cartridge includes a lancet receiving element configured to automatically cause the lancet to deploy the needle to draw a physiological sample upon engagement with the lancet.

10. The dermal patch system of claim 9, wherein the lancet is configured to automatically retract the needles.

11. The dermal patch system of claim 9, wherein the lancet receiving element includes the first electrical contacts and a portion of the lancet that engages with the lancet receiving element includes the second electrical contacts.

12. The dermal patch system of claim 4, wherein the power supply comprises a battery or a charged capacitor.

13. The dermal patch system of claim 1, wherein the power supply is a charged capacitor that automatically powers the biometric sensor and the computer system when the lancet engages with the cartridge.

14. The dermal patch system of claim 1, wherein the cartridge is configured to receive and store the physiological sample.

15. The dermal patch system of claim 1, wherein the biometric sensor is a fingerprint scanner or an iris scanner.

16. A method for obtaining a physiological sample from a subject, comprising:
affixing a cartridge of a dermal patch system to the skin of a subject,
engaging a lancet with the cartridge to draw a physiological sample, and
obtaining biometric data of the subject during at least a portion of temporal period associated with drawing the physiological.

17. The method of claim 16, further comprising:
storing the drawn physiological sample within the cartridge.

18. The method of claim 16, further comprising:
storing the fingerprint data in a memory of a computer system that is disposed within the cartridge.

19. The method of claim 16, wherein the cartridge includes a power supply that is configured to power the biometric sensor and the computer system upon engagement of the lancet with the cartridge.

20. The method of claim 19, wherein the power supply is a charged capacitor and engaging the lancet with the cartridge closes a circuit which causes the charged capacitor to automatically power the biometric sensor and the computer system.
